SEQUENCE LISTING (4 sequences)

SEQ ID NO: 1 - protein, 462 aa, Homo sapiens
  MOD_RES (56)..(56): PHOSPHORYLATION
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 2 - protein, 462 aa, Homo sapiens; identical to SEQ ID NO: 1

SEQ ID NO: 3 - protein, 12 aa, Homo sapiens
  MOD_RES (8)..(8): PHOSPHORYLATION
SPISTLSSPING

SEQ ID NO: 4 - protein, 12 aa, Homo sapiens
SPISTLSSPING

(19) United States
(12) Patent Application Publication
(10) Pub. No.: US 2024/0034785 A1
(43) Pub. Date: Feb. 1, 2024

(54) RXR-ALPHA BINDERS AND RXR-ALPHA/PLK1 MODULATORS

(71) Applicant: NUCMITO PHARMACEUTICALS CO. LTD., Xiamen, Fujian (CN)

(72) Inventors: Xiaokun Zhang, Xiamen (CN); Ying Su, Xiamen (CN); Guobin Xie, Xiamen (CN); Yuqi Zhou, Xiamen (CN)

(21) Appl. No.: 18/256,237

(22) PCT Filed: Dec. 7, 2020
(86) PCT No.: PCT/CN2020/134263

§ 371 Date: Jun. 7, 2023

(51) Int. Cl. C07K 16/28; G01N 33/574

(52) U.S. Cl. CPC C07K 16/28; G01N 33/57484; G01N 2333/70567

(57) ABSTRACT

Provided herein are retinoid X receptor alpha binders that specifically bind to an epitope of a retinoid X receptor alpha, wherein the epitope comprises a phosphorylated serine at position 56 or 70. Also provided herein are retinoid X receptor alpha/polo-like kinase 1 modulators that inhibit the interaction of a polo-like kinase 1 with a retinoid X receptor alpha comprising a phosphorylated serine at position 56 or 70.

### FIELD

Provided herein are retinoid X receptor alpha binders that specifically bind to an epitope of a retinoid X receptor alpha, wherein the epitope comprises a phosphorylated serine at position 56 or 70. Also provided herein are retinoid X receptor alpha/polo-like kinase 1 modulators that inhibit the interaction of a polo-like kinase 1 with a retinoid X receptor alpha comprising a phosphorylated serine at position 56 or 70.

### BACKGROUND

Retinoid X receptor alpha (RXRα), a unique member of the nuclear receptor superfamily of transcription factors, regulates a broad spectrum of physiological and pathological pathways, including cellular growth, proliferation, differentiation, and apoptosis. Dawson and Zhang, Curr. Med. Chem. 2002, 9, 623-37; Germain et al., Pharmacol. Rev. 2006, 58, 760-72; Liby et al., Nat. Rev. Cancer 2007, 7, 357-69; Altucci et al., Nat. Rev. Drug Discov. 2007, 6, 793-810; Su et al., Curr. Top. Med. Chein. 2017, 17, 663-75. RXRα contains three domains: a C-terminal ligand-binding domain (LBD) responsible for ligand-binding and receptor dimerization, a DNA-binding domain (DBD) for specific DNA-binding, and a structurally variable and plastic N-terminal A/B domain with a poorly defined function. Like other nuclear receptor family members, RXRα acts as a transcription factor to regulate target gene transcription by binding to its cognate DNA response elements either as a homodimer or heterodimer with another nuclear receptor family member. Zhang et al., Nature 1992, 355, 441-6; Kliewer et al., Nature 1992, 355, 446-9; Zhang et al., Nature 1992, 358, 587-91.

Disruption of normal RXRα signaling due to its altered expression and malfunction is implicated in the development of a number of malignancies. Altucci et al., Nat. Rev. Drug Discov. 2007, 6, 793-810; Su et al., Curr. Top. Med. Chem. 2017, 17, 663-75; Zhang et al., Acta Pharmacol. Sin. 2015, 36, 102-12. RXRα abnormalities including phosphorylation or proteolytic cleavage are frequently observed in tumor cells. Matsushima-Nishiwaki et al., Biochem. Biophys. Res. Commun. 1996, 225, 946-51; Nomura et al., Biochem. Biophys. Res. Commun. 1999, 254, 388-94; Zhong et al., Cancer Biol. Ther. 2003, 2, 179-84; Shimizu et al., Cancer Sci. 2009, 100, 369-74; Zhou et al., Cancer Cell 2010, 17, 560-73; Ye et al., Nat. Commun. 10, 2019, 1463. Consistent with its role in cancer development. RXRα is one of the important targets for the development of pharmacologic intervention and therapeutic applications. Dawson and Zhang, Curr. Med. Chem. 2002, 9, 623-37; Liby et al., Nat. Rev. Cancer 2007, 7, 357-69; Altucci et al., Nat. Rev. Drug Discov. 2007, 6, 793-810; de Lera et al., Nat. Rev. Drug Discov. 2007, 6, 811-20; Uray et al., Semin. Oncol. 2016, 43, 49-64; Su et al., Curr. Top. Med. Chem. 2017, 17, 663-75.

Mitosis, the most dynamic cell cycle phase that passes one of each pair of sister chromatids to each daughter cells, is orchestrated by a highly coordinated events. Cyclin-dependent kinase 1 (Cdk1) in complex with cyclin B1 controls the entry into mitosis from G2 phase of the cell cycle. Cdk1 acts in concert with polo-like kinase 1 (PLK1), another key mitotic kinase, to regulate critical mitotic events to ensure precise duplication of genetic materials. Barr et al., Nat. Rev. Mol. Cell Biol. 2004, 5, 429-40; Petronczki et al., Dev. Cell 2008, 14, 646-59; Archambault and Glover, Nat. Rev. Mol. Cell Biol. 2009, 10, 265-75; Zitouni et al., Nat. Rev. Mol. Cell Biol. 2014, 15, 433-452; Combes et al., Oncogene 2017, 36, 4819-27. The role of PLK1 is largely dependent on its localization to various subcellular structures during mitotic progression. At the centrosomes, the major microtubule-organizing centers (MTOCs) are crucial for the assembly of a bipolar mitotic spindle and subsequent faithful segregation of chromosomes into two daughter cells. Conduit et al., Nat. Rev. Mol. Cell Biol. 2015, 16, 611-24; Fu et al., Cold Spring Harb. Perspect. Biol. 2015, 7, a015800; Gonczy, Nat. Rev. Cancer 2015, 15, 639-52; Paz and Luders, Trends Cell Biol. 2018, 28, 176-87. PLK1 regulates centrosome maturation, disjunction, and microtubule attachment. While mitosis is highly regulated and coordinated in normal cells, it is exquisitely vulnerable to perturbations. Deregulated mitosis can result in tumorigenesis and/or rapid tumor cell proliferation. Both Cdk1 and PLK1 are abnormally activated in numerous tumor types and hence there has been intensive development of pharmacological inhibitors of Cdk1 and PLK1 for cancer therapy. McInnes et al., Nat. Chem. Biol. 2006, 2, 608-17; Strebhardt, Nat. Rev. Drug Discov. 2010, 9, 643-60; Asghar et al., Nat. Rev. Drug Discov. 2015, 14, 130-46; Dominguez-Brauer et al., Mol. Cell 2015, 60, 524-36; Otto and Sicinski, Nat. Rev. Cancer 2017, 17, 93-115.

Despite the advances in cancer treatment, cancer remains a major worldwide public health problem. It was estimated that there will be 1,806,590 new cancer cases diagnosed and 606,520 cancer deaths in the US alone in 2020. Cancer Facts & Figures 2020. Therefore, there is a need for an effective therapy for cancer treatment.

### SUMMARY OF THE DISCLOSURE

Provided herein is a retinoid X receptor alpha (RXRα) binder that specifically binds to an epitope of an RXRα, wherein the epitope comprises a phosphorylated serine at position 56 or 70.

Also provided herein is an RXRα binder that specifically binds to an epitope comprising amino acid residues 49 to 60 and a phosphorylated serine residue at position 56 as set forth in SEQ ID NO: 1.

Additionally, provided herein is an RXRα binder having a selectivity for a phosphorylated RXRα comprising a phosphorylated serine at position 56 over an RXRα comprising a unphosphorylated serine at position 56.

Furthermore, provided herein is an RXRα binder that specifically binds to a phosphopeptide comprising an amino acid sequence of SEQ ID NO: 3.

Provided herein is an RXRα binder having a selectivity for a phosphopeptide comprising an amino acid sequence of SEQ ID NO: 3 over a peptide comprising an amino acid sequence of SEQ ID NO: 4.

Provided herein is an immunogenic composition comprising a phosphopeptide that comprises an amino acid sequence of an epitope of an RXRα, wherein the epitope comprises a phosphorylated serine at position 56 or 70; and optionally an adjuvant.

Provided herein is an immunogenic composition comprising an epitope that comprises amino acid residues 49 to 60 and a phosphorylated serine residue at position 56 as set forth in SEQ ID NO: 1; and optionally an adjuvant.

Provided herein is an immunogenic composition comprising a phosphopeptide that comprises an amino acid sequence of SEQ ID NO: 3 and optionally an adjuvant.

Provided herein is a method of detecting a phosphorylated RXRα in a biological sample, comprising the steps of:
contacting the biological sample with an RXRα binder provided herein to form an RXRα binder/phosphorylated RXRα complex; and
detecting the RXRα binder/phosphorylated RXRα complex;
wherein the phosphorylated RXRα comprises a phosphorylated serine at position 56 or 70.

Provided herein is a method of diagnosing a proliferative disease in a subject by detecting the level of a phosphorylated RXRα in a biological sample from the subject, comprising the steps of:
contacting the biological sample with an RXRα binder provided herein to form an RXRα binder/phosphorylated RXRα complex; and
detecting the RXRα binder/phosphorylated RXRα complex;
wherein the phosphorylated RXRα comprises a phosphorylated serine at position 56 or 70.

Provided herein is a method for screening a subject for a proliferative disease by detecting the level of a phosphorylated RXRα in a biological sample from the subject, comprising the steps of:
contacting the biological sample with an RXRα binder provided herein to form an RXRα binder/phosphorylated RXRα complex; and
detecting the RXRα binder/phosphorylated RXRα complex;
wherein the phosphorylated RXRα comprises a phosphorylated serine at position 56.

Provided herein is a method of treating, preventing, or ameliorating one or more symptoms of a proliferative disease in a subject, comprising administering a therapeutically effective amount of a retinoid X receptor alpha/polo-like kinase 1 (RXRα/PLK1) modulator that inhibits the interaction of a PLK1 with an RXRα comprising a phosphorylated serine at position 56 or 70.

Provided herein is a method of inhibiting the growth of a cell, comprising contacting the cell with an effective amount of an RXRα/PLK1 modulator that inhibits the interaction of a PLK1 with an RXRα comprising a phosphorylated serine at position 56 or 70.

Provided herein is a method of inducing apoptosis in a cell, comprising contacting the cell with an effective amount of an RXRα/PLK1 modulator that inhibits the interaction of a PLK1 with an RXRα comprising a phosphorylated serine at position 56 or 70.

Provided herein is a method of inhibiting mitotic progression in a cell, comprising contacting the cell with an effective amount of an RXRα/PLK1 modulator that inhibits the interaction of a PLK1 with an RXRα comprising a phosphorylated serine at position 56 or 70.

### BRIEF DESCRIPTION OF THE DRAWINGS

FIG. 1 shows the analysis of the interaction of a transfected Myc-RXRα with FLAG-PLK1 or FLAG-RARγ using a co-immunoprecipitation (CoIP) assay after HeLa cells were treated with or without 9-cis-RA (0.1 μM) for 3 hr, where immunoprecipitates were analyzed by western blot (WB).

FIG. 2 shows the structures of RXRα, PLK1, and mutants, where DBD represents a DNA-binding domain; LBD represents a ligand-binding domain; KD represents a kinase domain; PB represents a polo-box; and PBD represents a PB domain.

FIG. 3 shows the CoIP analysis of the interaction of RXRα-1-235 with PLK1 in HeLa cells.

FIG. 4 shows the CoIP analysis of the interaction of RXRα-LBD with PLK1 in HeLa cells.

FIG. 5 shows the CoIP analysis of the interaction of RXRα-AA/B with PLK1 in HeLa cells.

FIG. 6 shows the CoIP analysis of the interaction of PLK1-KD or PLK1-PBD with RXRα in HeLa cells, where SE represents short-time exposure; and LE represents long-time exposure.

FIG. 7 shows the WB analysis of HeLa cells released from a double thymidine (TT) block for the indicated time, where cell cycle distribution was determined by fluorescence activated cell sorting (FACS) and AS represents asynchronous cells.

FIG. 8 shows the WB analysis of HeLa cells treated with nocodazole (50 ng/mL).

FIG. 9 shows the WB analysis of RXRα modification in mice subjected to liver PH.

FIG. 10 shows the CoIP analysis of the interaction of endogenous m-RXRα with PLK1 in HeLa cells released from a TT block for 10 hr.

FIG. 11 shows the CoIP analysis of the interaction of endogenous m-RXRα with PLK1 in HepG2 cells released from a nocodazole block for 1 hr.

FIG. 12 shows the CoIP analysis of HeLa cells transfected with or without FLAG-RXRα and released from a TT block using an anti-FLAG antibody.

FIG. 13 shows the CoIP analysis of HeLa cells transfected with or without FLAG-PLK1 and released from a TT block using an anti-FLAG antibody.

FIG. 14 shows the WB analysis of TAP effect on the stability of m-RXRα expressed in mitotic HeLa cells.

FIG. 15 shows the WB analysis of transfected FLAG-RXRα immunoprecipitated from mitotic HeLa cells using an anti-P-Ser or anti-P-Thr antibody.

FIG. 16 shows the WB analysis of HeLa cells released from a TI block and treated with the indicated inhibitors for 15 min.

FIG. 17 shows the WB analysis of purified GST-RXRα subjected to phosphorylation by FLAG-Cdk1/Myc-cyclin B1 with or without Cdk1 inhibitor RO-3306.

FIG. 18 shows the WB analysis of HeLa cells transfected with FLAG-RXRα and increasing concentration of Flag-Cdk1 and Myc-cyclin B1.

FIG. 19 shows the CoIP analysis of HeLa cells transfected with HA-RXRα and FLAG-Cdk1 for their interaction.

FIG. 20 shows the CoIP analysis of the interaction of the indicated PLK1 mutants with p-RXRα in HeLa cells.

FIG. 21 shows the WB analysis of HeLa cells transfected with the indicated RXRα or mutants released from a TT block.

FIG. 22 shows the WB analysis of HeLa cells transfected with the indicated RXRα or mutants released from a TT block.

FIG. 23 shows the CoIP analysis of the interaction of the indicated RXRα mutants with PLK1 in HeLa cells.

FIG. 24 shows the WB analysis of HeLa cells transfected with the indicated RXRα or mutants released from a TT block.

FIG. 25 shows the MS/MS analysis of p-RXRα phosphorylation sites.

FIG. 26 shows the WB analysis of GST-RXRα or GST-RXRα-S56A/S70A(2A) incubated with FLAG-Cdk1/Myc-cyclin B1.

FIG. 27 shows the CoIP analysis of HeLa cells transfected with FLAG-PLK1 and Myc-RXRα or mutants for their interaction.

FIG. 28 shows the alignment of RXRα protein sequences harboring Cdk1-phosphorylation sites/PLK1-interacting motifs from different species.

FIG. 29 shows the characterization of pS56-RXRα antibody on peptide spot arrays.

FIG. 30 shows the WB analysis of HeLa cells released from a TT block for the indicated time using an anti-pS56-RXRα antibody and other indicated antibodies.

FIG. 31 shows the WB analysis of mitotic HeLa cells transfected with an RXRα siRNA.

FIG. 32 shows the WB analysis of centrosomal fractions collected after sucrose density centrifugation of lysates prepared from mitotic HeLa cells.

FIG. 33 shows the quantitative analysis of the effect of the Cdk1 inhibitor RO-3306 on the centrosomal localization of pS56-RXRα or RXRα, where fluorescence intensity of pS56-RXRα or RXRα at 30 centrosomes in metaphase HeLa cells were analyzed (*SEM; ***p<0.001).

FIG. 34 shows the WB analysis of HeLa cells transfected with an RXRα siRNA or PLK1 siRNA for 48 hr.

FIG. 35 shows the WB analysis of HeLa cells released for 10 hr, where, after transfected with RXRα-r or RXRα-2A-r and synchronized by TT treatment, the cells were transfected again with RXRα siRNAs during the second thymidine arrest.

FIG. 36 shows the analysis of HeLa cells released from a TT block for 10 hr after transfected with a control siRNA or RXRα siRNA, where relative fluorescence intensity of PLK1-pT210 at the centrosome during metaphase was analyzed with at least 40 centrosomes (±SEM; **p<0.01).

FIG. 37 shows the analysis of HeLa cells transfected with the indicated expression vector, where relative fluorescence intensity of PLK1-pT210 at the centrosome was analyzed with at least 30 centrosomes (±SEM; ns, not significant; *p<0.05).

FIG. 38 shows the WB analysis of GST-RXRα and GST-RXRα-2A phosphorylation, where they were subjected to phosphorylation in vitro by FLAG-Cdk1/Myc-cyclin B1, immunoprecipitated from mitotic HeLa cells, and then incubated with His-Aurora A and His-PLK1 after removing FLAG-Cdk1/Myc-cyclin B1.

FIG. 39 shows the CoIP analysis of HeLa cells released from a TT block for 10 hr after transfected with FLAG-Aurora A.

FIG. 40 shows the analysis of HeLa cells released from a TT block for 10 hr after transfected with a control siRNA or RXRα siRNA, where relative fluorescence intensity of γ-tubulin at the centrosome during prophase was scored with at least 30 centrosomes (±SEM; *p<0.05).

FIG. 41 shows the analysis of HeLa cells after the cells transfected with a control siRNA or RXRα siRNA were cold-treated and reheated for the indicated time at 37° C., where relative MT nucleation activity at the centrosomes during prophase was scored (n=40 cells) (±SEM; ***p<0.001).

FIGS. 42A and 42B show the analysis of HeLa cells released from a TT block for 10 hr after transfected with a control siRNA or RXRα siRNA, where the percentage of cells with chromosome misalignment, multipolar spindle and multicentrosomes was calculated by counting 500 cells (±SEM; *p<0.05).

FIG. 43 shows the IF analysis of RXRα/HeLa cells after transfected with FLAG-RXRα and mutants for 24 hr, where the percentage of cells with chromosome misalignment was calculated by counting 300 cells (±SEM; *p<0.05; ***p<0.001).

FIG. 44 shows the FACS analysis of HeLa cells released for the indicated time after synchronized by TT treatment and transfected with an RXRα siRNAs or PLK1 siRNA during the second thymidine arrest, where the percentage of cells with 2N and 4N is shown.

FIG. 45 shows the WB analysis of lysates prepared from primary normal liver and primary liver tumor cells from different mice.

FIG. 46 shows the CoIP analysis of the interaction of endogenous p-RXRα with PLK1 in primary mouse normal liver and primary mouse liver tumor cells.

FIG. 47 shows the WB analysis of MEF, melanoma B16F10 cells, and breast cancer 4T1 cells.

FIG. 48 shows the cell cycle profiles of MEF, B16F10, and 4T1 cells.

FIG. 49 shows CoIP analysis of the interaction of m-RXRα with PLK1 in MEF, B16F10 or 4T1 cells.

FIG. 50 shows the analysis of m-RXRα and PLK1 in MEF, B16F10 or 4T1 cells for their colocalization, where relative fluorescence intensity of pS56-RXRα at the centrosome during metaphase was analyzed with at least 50 centrosomes (±SEM; ***p<0.001).

FIG. 51 shows the WB analysis of HepG2 liver tumor and THLE-2 noncancerous liver cells.

FIG. 52 shows the WB analysis of liver tissues from control mice and liver tumor tissues from mice treated with CCl₄/DEN.

FIG. 53 shows the WB analysis of pS56-RXRα expression in human liver cancer tissues (T) and their corresponding tumor adjacent normal tissues (N).

FIG. 54 shows the Kaplan-Meier plot of overall survival of patients with HCC stratified by negative pS56-RXRα or positive pS56-RXRα expression levels, where a log-rank test is used for statistical analysis.

FIG. 55 shows the CoIP analysis of HeLa cells transfected with Myc-RXRα and FLAG-PLK1 or FLAG-RARγ, where the cells were treated with or without compound A1 (10 μM) for 3 hr.

FIG. 56 shows the analysis of HepG2 cells that were treated with RO-3306 (10 μM) for 30 min or compound A1 (10 μM) for 2 hr, where PLA was used to detect RXRα interaction with PLK1 at the centrosome and the percentage of PLA⁺ cells during metaphase was calculated by counting 200 cells (±SEM; ***p<0.001).

FIG. 57 shows the analysis of HepG2 cells that were treated with compound A1 (10 μM) for 2 hr after released from a TT block for 10 hr, where relative fluorescence intensity of PLK1-pT210 at the centrosomes in metaphase cells was scored with at least 50 centrosomes (±SEM; **p<0.01).

FIG. 58 shows the analysis of HepG2 cells that were treated with compound A1 (10 μM) for 2 hr after released from a TT block for 10 hr, where relative fluorescence intensity of γ-tubulin at the centrosomes in metaphase HepG2 cells was scored with at least 50 centrosomes (±SEM; ***p<0.001).

FIG. 59 shows the analysis of HeLa cells that were cold-treated, reheated for indicated time at 37° C. in the presence or absence of compound A1 (10 μM), where relative MT nucleation activity at the centrosomes during prophase is shown (n=40 cells) (±SEM; ***p<0.001).

FIG. 60 shows the FACS analysis of the indicated cell lines treated with compound A1 (10 M) for 12 hr.

FIG. 61 shows the WB analysis of liver cancer HepG2 cells and QSG-7701 normal liver cells treated with indicated concentration of compound A1 for 12 hr.

FIG. 62 shows the WB analysis of liver cancer Bel-7402 and SK-Hep-1 treated with compound A1 (10 μM) or BI2536 (0.25 μM) for 12 hr.

FIG. 63 shows the WB analysis of primary hepatic carcinoma cells from a liver cancer patient treated with compound A1 (10 μM) or BI2536 (0.25 μM) for 48 hr.

FIG. 64 shows the WB analysis of asynchronous HeLa cells (AS) or HeLa cells synchronized at the G1/S transition by thymidine treatment, where the cells were incubated with compound A1 (10 μM) for 24 hr.

FIG. 65 shows the WB analysis of primary mouse normal liver or liver tumor cells, which were treated with compound A1 (10 μM) for 24 hr.

FIG. 66 shows the WB analysis of MEF, B16F10, and 4T1 cells, where the cells were treated with compound A1 (10 μM) for 6 hr.

FIG. 67 shows the analysis of MEF, B16F10, and 4T1 cells, where the cells were treated with compound A1 (10 μM) for 2 hr, and fixed and stained for DAPI (blue), and where the percentage of cells with chromosome misalignment during metaphase was calculated by counting at least 300 cells (±SEM; ns, not significant; ***p<0.001).

FIG. 68 shows the WB analysis of HepG2 and THLE-2 cells that were treated with compound A1 (10 μM) or B12536 (0.25 μM) for 12 hr.

FIGS. 69 and 70 show the analysis of nude mice injected with HepG2 cells, where the mice were treated with compound A1 (80 mg/kg) once every two days. The tumor volume was monitored and recorded. Tumors excised at day 14 were shown in FIG. 69 and weight in FIG. 70 (±SEM; **p<0.01).

FIG. 71 shows the WB analysis of tumor specimens or normal liver tissues from HepG2 xenograft of mice treated with or without compound A1.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, immunology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare et al., Eds.; Academic Press: London, 2020; Handbook of Pharmaceutical Excipients, 9th ed.; Sheskey et al., Eds.; Pharmaceutical Press: London, 2020; Handbook of Pharmaceutical Additives, 3rd ed.; Ash and Ash Eds.; Synapse Information Resources: 2007; Pharmaceutical Preformulation and Formulation, 2nd ed.; Gibson Ed.; Drugs and the Pharmaceutical Sciences 199; Informa Healthcare: New York, NY, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵CI), chlorine-36 (³⁶Cl), chlorine-37 (³⁷Cl), bromine-79 (⁷⁹Br), bromine-81 (⁸¹Br), iodine-123 (¹²I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²¹I), iodine-129 (¹²⁹I), and iodine-131 (¹³¹I). It will be understood that, in a compound as described herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound described herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

When a compound described herein contains an acidic or basic moiety, it can be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011.

Suitable acids for use in the preparation of pharmaceutically acceptable salts of a compound described herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound described herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

RXRα Binders

In one embodiment, provided herein is a retinoid X receptor alpha (RXRα) binder that specifically binds to an epitope of an RXRα, wherein the epitope comprises a phosphorylated serine at position 56 or 70.

In certain embodiments, the RXRα is a human RXRα. In certain embodiments, the RXRα has an amino acid sequence of SEQ ID NO: 1.

In certain embodiments, the epitope is a linear epitope. In certain embodiments, the linear epitope has a length ranging from about 5 to about 50, from about 5 to about 25, or from about 10 to 20 amino acids. In certain embodiments, the linear epitope has a length ranging from about 5 to about 50 amino acids. In certain embodiments, the linear epitope has a length ranging from about 5 to about 25 amino acids. In certain embodiments, the linear epitope has a length ranging from about 10 to about 20 amino acids. In certain embodiments, the linear epitope has a length of about 8, about 9, about 10, about 11, about 12, about 13, about 14, about 15, about 16, about 17, about 18, about 19, or about 20 amino acids.

In certain embodiments, the epitope comprises a phosphorylated serine at position 56 or 70 as set forth in SEQ ID NO: 1. In certain embodiments, the epitope comprises a phosphorylated serine at position 56 as set forth in SEQ ID NO: 1. In certain embodiments, the epitope comprises a phosphorylated serine at position 70 as set forth in SEQ ID NO: 1.

In certain embodiments, the linear epitope has a length ranging from 5 to 25 amino acids and comprises a phosphorylated serine at position 56 or 70 as set forth in SEQ ID NO: 1. In certain embodiments, the linear epitope has a length ranging from 5 to 25 amino acids and comprises a phosphorylated serine at position 56 as set forth in SEQ ID NO: 1. In certain embodiments, the linear epitope has a length ranging from 5 to 25 amino acids and comprises a phosphorylated serine at position 70 as set forth in SEQ ID NO: 1.

In certain embodiments, the linear epitope having a length of about 10, about 11, about 12, about 13, about 14, or about 15 amino acids; and comprises a phosphorylated serine at position 56 or 70 as set forth in SEQ ID NO: 1. In certain embodiments, the linear epitope has a length of about 10, about 11, about 12, about 13, about 14, or about 15 amino acids; and comprises a phosphorylated serine at position 56 as set forth in SEQ ID NO: 1. In certain embodiments, the linear epitope having a length of about 10, about 11, about 12, about 13, about 14, or about 15 amino acids; and comprises a phosphorylated serine at position 70 as set forth in SEQ ID NO: 1.

In certain embodiments, the linear epitope comprises an amino acid sequence that is no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 3. In certain embodiments, the linear epitope comprises an amino acid sequence that is no less than about 80% identical to the amino acid sequence of SEQ ID NO: 3. In certain embodiments, the linear epitope comprises an amino acid sequence that is no less than about 85% identical to the amino acid sequence of SEQ ID NO: 3. In certain embodiments, the linear epitope comprises an amino acid sequence that is no less than about 90% identical to the amino acid sequence of SEQ ID NO: 3. In certain embodiments, the linear epitope comprises an amino acid sequence that is no less than about 91% identical to the amino acid sequence of SEQ ID NO: 3. In certain embodiments, the linear epitope comprises an amino acid sequence that is no less than about 92% identical to the amino acid sequence of SEQ ID NO: 3. In certain embodiments, the linear epitope comprises an amino acid sequence that is no less than about 93% identical to the amino acid sequence of SEQ ID NO: 3. In certain embodiments, the linear epitope comprises an amino acid sequence that is no less than about 94% identical to the amino acid sequence of SEQ ID NO: 3. In certain embodiments, the linear epitope comprises an amino acid sequence that is no less than about 95% identical to the amino acid sequence of SEQ ID NO: 3. In certain embodiments, the linear epitope comprises an amino acid sequence that is no less than about 96% identical to the amino acid sequence of SEQ ID NO: 3. In certain embodiments, the linear epitope comprises an amino acid sequence that is no less than about 97% identical to the amino acid sequence of SEQ ID NO: 3. In certain embodiments, the linear epitope comprises an amino acid sequence that is no less than about 98% identical to the amino acid sequence of SEQ ID NO: 3. In certain embodiments, the linear epitope comprises an amino acid sequence that is no less than about 99% identical to the amino acid sequence of SEQ ID NO: 3.

In another embodiment, provided herein is an RXRα binder that specifically binds to an epitope comprising amino acid residues 49 to 60 and a phosphorylated serine residue at position 56 as set forth in SEQ ID NO: 1.

In yet another embodiment, provided herein is an RXRα binder comprising a phosphorylated serine at position 56 over an RXRα comprising a unphosphorylated serine at position 56.

In certain embodiments, an RXRα binder provided herein has a selectivity for a phosphorylated RXRα comprising an amino acid sequence of SEQ ID NO: 1 over an RXRα comprising an amino acid sequence of SEQ ID NO: 2.

In certain embodiments, an RXRα binder provided herein has a selectivity for a phosphorylated RXRα comprising a phosphorylated serine at position 56 over an RXRα comprising a unphosphorylated serine at position 56, wherein the selectivity is no greater than about 0.1, no greater than about 0.01, or no greater than about 0.001; and wherein the selectivity is measured as a ratio of a dissociation constant (K_{d}) of the RXRα binder to the RXRα with a phosphorylated serine 56 over a dissociation constant (K_{d}) of the RXRα binder to the RXRα with an unphosphorylated serine 56.

In yet another embodiment, provided herein is an RXRα binder that specifically binds to a phosphopeptide comprising an amino acid sequence of SEQ ID NO: 3.

In still another embodiment, provided herein is an RXRα binder having a selectivity for a phosphopeptide comprising an amino acid sequence of SEQ ID NO: 3 over a peptide comprising an amino acid sequence of SEQ ID NO: 4.

In certain embodiments, an RXRα binder provided herein has a selectivity for the phosphopeptide of SEQ ID NO: 3 over the unphosphorylated peptide of SEQ ID NO: 4, wherein the selectivity is no greater than about 0.1, no greater than about 0.01, or no greater than about 0.001; and wherein the selectivity is measured as a ratio of a dissociation constant (K_{d}) of the RXRα binder to the phosphopeptide over a dissociation constant (K_{d}) of the RXRα binder to the unphosphorylated peptide.

In one embodiment, the RXRα binder is an antibody or an antigen-binding fragment thereof. In another embodiment, the RXRα binder is an IgA, IgD, IgE, IgG, or IgM antibody, or an antigen-binding fragment thereof. In yet another embodiment, the RXRα binder is an IgA antibody or an antigen-binding fragment thereof. In yet another embodiment, the RXRα binder is an IgD antibody or an antigen-binding fragment thereof. In yet another embodiment, the RXRα binder is an IgE antibody or an antigen-binding fragment thereof. In yet another embodiment, the RXRα binder is an IgG antibody or an antigen-binding fragment thereof. In still another embodiment, the RXRα binder is an IgM antibody or an antigen-binding fragment thereof.

In one embodiment, the RXRα binder is an IgA1, IgA2, IgG1, IgG2, IgG3, or IgG4 antibody, or an antigen-binding fragment thereof. In another embodiment, the RXRα binder is an IgA1 or IgA2, or an antigen-binding fragment thereof. In yet another embodiment, the RXRα binder is an IgA1 or an antigen-binding fragment thereof. In yet another embodiment, the RXRα binder is an IgA2 or an antigen-binding fragment thereof. In yet another embodiment, the RXRα binder is an IgG1, IgG2, IgG3, or IgG4 antibody, or an antigen-binding fragment thereof. In yet another embodiment, the RXRα binder is an IgGI antibody or an antigen-binding fragment thereof. In yet another embodiment, the RXRα binder is an IgG2 antibody or an antigen-binding fragment thereof. In yet another embodiment, the RXRα binder is an IgG3 antibody or an antigen-binding fragment thereof. In still another embodiment, the RXRα binder is an IgG4 antibody or an antigen-binding fragment thereof.

In one embodiment, the RXRα binder is a single-chain variable fragment (scFv), Fab, Fab′, F(ab)₂, F(ab′)₂, Fv, diabody, triabody, tetrabody, or minibody. In another embodiment, the RXRα binder is an scFv. In yet another embodiment, the RXRα binder is a Fab. In yet another embodiment, the RXRα binder is a Fab′. In yet another embodiment, the RXRα binder is a F(ab)₂. In yet another embodiment, the RXRα binder is a F(ab′)₂. In yet another embodiment, the RXRα binder is a Fv. In yet another embodiment, the RXRα binder is a diabody. In yet another embodiment, the RXRα binder is a triabody. In yet another embodiment, the RXRα binder is a tetrabody. In still another embodiment, the RXRα binder is a minibody.

In one embodiment, the RXRα binder is synthetic or recombinant. In another embodiment, the RXRα binder is purified. In yet another embodiment, the RXRα binder is isolated.

In one embodiment, the RXRα binder binds to the RXRα of SEQ ID NO: 1 with a K_{d} ranging from about 1 μM to about 1,000 nM, from about 10 μM to about 200 nM, or from about 100 μM to about 100 nM. In another embodiment, the RXRα binder binds to the RXRα of SEQ ID NO: 1 with a K_{d} ranging from about 1 μM to about 1,000 nM. In yet another embodiment, the RXRα binder binds to the RXRα of SEQ ID NO: 1 with a K_{d} ranging from about 10 μM to about 200 nM. In still another embodiment, the RXRα binder binds to the RXRα of SEQ ID NO: 1 with a K_{d} ranging from about 100 μM to about 100 nM.

In one embodiment, the RXRα binder binds to a phosphopeptide of SEQ ID NO: 3 with a K_{d} ranging from about 1 μM to about 1,000 nM, from about 10 μM to about 200 nM, or from about 100 μM to about 100 nM. In another embodiment, the RXRα binder binds to a phosphopeptide of SEQ ID NO: 3 with a K_{d} ranging from about 1 μM to about 1,000 nM. In yet another embodiment, the RXRα binder binds to a phosphopeptide of SEQ ID NO: 3 with a K_{d} ranging from about 10 μM to about 200 nM. In still another embodiment, the RXRα binder binds to a phosphopeptide of SEQ ID NO: 3 with a K_{d} ranging from about 100 μM to about 100 nM.

In certain embodiments, the RXRα binder is a monoclonal antibody. In certain embodiments, the RXRα binder is a polyclonal antibody. In certain embodiments, the antibody is a human, humanized, or chimeric antibody.

In certain embodiments, the RXRα binder is a chicken, donkey, goat, guinea pig, hamster, mouse, rabbit, rat, or sheep antibody, or an antigen-binding fragment thereof. In certain embodiments, the RXRα binder is a chicken antibody, or an antigen-binding fragment thereof. In certain embodiments, the RXRα binder is a donkey antibody, or an antigen-binding fragment thereof. In certain embodiments, the RXRα binder is a goat antibody, or an antigen-binding fragment thereof. In certain embodiments, the RXRα binder is a guinea pig antibody, or an antigen-binding fragment thereof. In certain embodiments, the RXRα binder is a hamster antibody, or an antigen-binding fragment thereof. In certain embodiments, the RXRα binder is a mouse antibody, or an antigen-binding fragment thereof. In certain embodiments, the RXRα binder is a rabbit antibody, or an antigen-binding fragment thereof. In certain embodiments, the RXRα binder is a rat antibody, or an antigen-binding fragment thereof. In certain embodiments, the RXRα binder is a sheep antibody, or an antigen-binding fragment thereof.

In certain embodiments, the RXRα binder comprises a reporter. In certain embodiments, the reporter is a chromogenic reporter. In certain embodiments, the chromogenic reporter is an enzyme. In certain embodiments, the chromogenic reporter is a peroxidase. In certain embodiments, the chromogenic reporter is a horseradish peroxidase or alkaline peroxidase. In certain embodiments, the chromogenic reporter is a horseradish peroxidase. In certain embodiments, the chromogenic reporter is an alkaline peroxidase.

In certain embodiments, the RXRα binder is an enzyme conjugated secondary antibody or an antigen-binding fragment thereof. In certain embodiments, the RXRα binder is an antibody or an antigen-binding fragment thereof, conjugated with a peroxidase. In certain embodiments, the RXRα binder is an antibody or an antigen-binding fragment thereof, conjugated with a horseradish peroxidase or alkaline peroxidase. In certain embodiments, the RXRα binder is an antibody or an antigen-binding fragment thereof, conjugated with a horseradish peroxidase. In certain embodiments, the RXRα binder is an antibody or an antigen-binding fragment thereof, conjugated with an alkaline peroxidase.

In certain embodiments, the enzyme conjugated antibody described herein requires a substrate to generate a signal for detection. In certain embodiments, the substrate is a colorimetric substrate. In certain embodiments, the substrate is a fluorescent substrate. In certain embodiments, the substrate is a chemiluminescent substrate. In certain embodiments, the substrate is an electrochemiluminescent substrate. Suitable substrates for a horseradish peroxidase include, but are not limited to, 2,2′-azinobis(3-ethylbenzothiazoline-6-sulfonic acid) diammonium salt (ABTS), luminol, O-phenylenediamine dihydrochloride (OPD), 3,3′,5,5′-tetramethylbenzidine (TMB), AMPLEX™ RED, AMPLEX™ ULTRARED, QUANTABLU™, QUANTARED™, and SUPERSIGNAL™. Suitable substrates for an alkaline peroxidase include, but are not limited to, adamantyl 1, 2-dioxetane aryl phosphate (AMPPD), p-nitrophenyl phosphate (PNPP), CDP-STAR™, CSPD™, and DYNALIGHT™.

In certain embodiments, the reporter is a colorimetric reporter. In certain embodiments, the reporter is a color particle. In certain embodiments, the reporter is a color microparticle or microsphere. In certain embodiments, the reporter is a color nanoparticle. In certain embodiments, the reporter is a gold particle. In certain embodiments, the reporter is a gold microparticle. In certain embodiments, the is a gold nanoparticle. In certain embodiments, the reporter is a colloidal gold nanoparticle. In certain embodiments, the reporter is a colored latex particle. In certain embodiments, the reporter is a colored latex microparticle. In certain embodiments, the reporter is a colored latex nanoparticle. In certain embodiments, the reporter is a colored polystyrene particle. In certain embodiments, the reporter is a colored polystyrene microparticle. In certain embodiments, the reporter is a colored polystyrene nanoparticle.

In certain embodiments, the reporter is a fluorescent reporter. In certain embodiments, the reporter is an organic fluorophore. In certain embodiments, the reporter is a fluorescent protein. Suitable fluorescent reporters include, but are not limited to, a cyanine dye (e.g., Cy2, Cy3, Cy3B, Cy3.5, Cy5, Cy5.5, and Cy7), a fluorescein dye (e.g., fluorescein isothiocyanate (FITC) and fluorescein diacetate), a rhodamine dye (e.g., rhodamine 6G, rhodamine 123, rhodamine B, rhodamine red, sulforhodamine B (SRB), sulforhodamine 101 (TEXAS RED™), carboxytetramethylrhodamine (TAMRA), tetramethylrhodamine (TMR), and tetramethylrhodamine isothiocyanate (TRITC)), an ALEXA FLUOR™ dye (e.g., ALEXA FLUOR™ 405, ALEXA FLUOR™ 530, ALEXA FLUOR™ 488, ALEXA FLUOR™ 500, ALEXA FLUOR™ 514, ALEXA FLUOR™ 532, ALEXA FLUOR™ 546, ALEXA FLUOR™ 555, ALEXA FLUOR™ 568, ALEXA FLUOR™ 594, ALEXA FLUOR™ 610, ALEXA FLUOR™ 633, ALEXA FLUOR™ 635. ALEXA FLUOR™ 647, ALEXA FLUOR™660, ALEXA FLUOR™ 680, ALEXA FLUOR™700, ALEXA FLUOR™750, and ALEXA FLUOR™ 790), a DYLIGHT™ FLUOR dye (e.g., DYLIGHT™ FLUOR 350, DYLIGHT™ FLUOR 405, DYLIGHT™ FLUOR 488. DYLIGHT™ FLUOR 550. DYLIGHT™ FLUOR 594, DYLIGHT™ FLUOR 633, DYLIGHT™ FLUOR 650. DYLIGHT™ FLUOR 680. DYLIGHT™ FLUOR 755, and DYLIGHT™ FLUOR 800), blue fluorescent protein (BFP), cyan fluorescent protein (CFP), green fluorescent protein (GFP), yellow fluorescent protein (YFP), a phycobiliprotein (e.g., allophycoerythrin, B-phycoerythrin, C-phycoerythrin, or R-phycoerythrin), and a quantum dot.

In certain embodiments, the reporter is a chemiluminescent reporter. In certain embodiment, the chemiluminescent reporter is an acridinium or ruthenium ester. In certain embodiments, the reporter is an electrochemiluminescent reporter. In certain embodiments, the electrochemiluminescent reporter is tris(2,2′-bipyridyl)ruthenium (II) chloride or dichlorotris(1,10-phenanthroline)ruthenium (II).

In certain embodiments, the reporter is a radioactive reporter. In certain embodiments, the radioactive reporter is a ³H, ¹²⁵I, ⁵S, ¹⁴C, ³²P, or ³³P containing reporter.

In one embodiment, provided herein is an immunogenic composition comprising a phosphopeptide that comprises an amino acid sequence of an epitope of an RXRα, wherein the epitope comprises a phosphorylated serine at position 56 or 70; and optionally an adjuvant.

In another embodiment, provided herein is an immunogenic composition comprising an epitope that comprises amino acid residues 49 to 60 and a phosphorylated serine residue at position 56 as set forth in SEQ ID NO: 1; and optionally an adjuvant.

In yet another embodiment, provided herein is an immunogenic composition comprising a phosphopeptide that comprises an amino acid sequence of SEQ ID NO: 3 and optionally an adjuvant.

In certain embodiments, the adjuvant suitable for an immunogenic composition provided herein is QS-21, Detox-PC, MPL-SE, MoGM-CSF, TiterMax-G, CRL-1005, GERBU, TERamide, PSC97B, Adjumer, PG-026, GSK-I, GcMAF B-alethine, MPC-026, Adjuvax, CpG ODN, Betafectin, alum, or MF59. In certain embodiments, the adjuvant suitable for an immunogenic composition provided herein is a lectin, a growth factor, cytokine, or lymphokine. In certain embodiments, the adjuvant suitable for an immunogenic composition provided herein is interferon-α, interferon-γ, a platelet derived growth factor (PDGF), a granulocyte-colony stimulating factor (gCSF), a granulocyte macrophage colony stimulating factor (gMCSF), a tumor necrosis factor (TNF), an epidermal growth factor (EGF), interleukin-1, interleukin-2, interleukin-4, interleukin-6, interleukin-8, interleukin-10, or interleukin-12. In certain embodiments, the adjuvant suitable for an immunogenic composition provided herein is aluminum hydroxide, aluminum phosphate, alum (potassium aluminum sulfate), monophosphoryl lipid A (MPL), polysorbate 80, squalene, vitamin E. AS01B, AS03, AS04, CpG 1018, MF59, or QS-21.

In one embodiment, provided herein is a method of detecting the level of a phosphorylated RXRα in a biological sample, comprising the steps of:
contacting the biological sample with an RXRα binder provided herein to form an RXRα binder/phosphorylated RXRα complex; and
detecting the RXRα binder/phosphorylated RXRα complex;
wherein the phosphorylated RXRα comprises a phosphorylated serine at position 56 or 70.

In another embodiment, provided herein is a method of diagnosing a proliferative disease in a subject by detecting the level of a phosphorylated RXRα in a biological sample from the subject, comprising the steps of:
contacting the biological sample with an RXRα binder provided herein to form an RXRα binder/phosphorylated RXRα complex; and
detecting the RXRα binder/phosphorylated RXRα complex;
wherein the phosphorylated RXRα comprises a phosphorylated serine at position 56 or 70.

In yet another embodiment, provided herein is a method for screening a subject for a proliferative disease by detecting the level of a phosphorylated RXRα in a biological sample from the subject, comprising the steps of:
contacting the biological sample with an RXRα binder provided herein to form an RXRα binder/phosphorylated RXRα complex; and
detecting the RXRα binder/phosphorylated RXRα complex;
wherein the phosphorylated RXRα comprises a phosphorylated serine at position 56 or 70.

In certain embodiments, the phosphorylated RXRα comprises a phosphorylated serine at position 56. In certain embodiments, the phosphorylated RXRα comprises a phosphorylated serine at position 70.

In certain embodiments, the phosphorylated RXRα comprises a phosphorylated serine at position 56 or 70 as set forth in SEQ ID NO: 1. In certain embodiments, the phosphorylated RXRα comprises a phosphorylated serine at position 56 as set forth in SEQ ID NO: 1. In certain embodiments, the phosphorylated RXRα comprises a phosphorylated serine at position 70 as set forth in SEQ ID NO: 1.

In certain embodiments, the phosphorylated RXRα is a human phosphorylated RXRα. In certain embodiments, the phosphorylated RXRα comprises an amino acid sequence of SEQ ID NO: 1.

In certain embodiments, the subject is a mammalian subject. In certain embodiments, the subject is a human.

In certain embodiments, the biological sample is a bodily fluid. In certain embodiments, the biological sample is a blood, plasma, serum, cerebral spinal fluid, mucus, saliva, semen, sputum, stool, or urine sample. In certain embodiments, the biological sample is a tissue (e.g., a tissue homogenate) or a cell lysate. In certain embodiments, the biological sample is a biopsy of a tissue.

In certain embodiments, a method provided herein further comprises a step of preparing the biological sample for analysis.

In certain embodiments, the detecting step is performed visually. In certain embodiments, the detecting step is performed colorimetrically. In certain embodiments, the detecting step is performed fluorescently. In certain embodiments, the detecting step is performed by chemiluminescence. In certain embodiments, the detecting step is performed by electrochemiluminescence. In certain embodiments, the detecting step is performed radioactively. In certain embodiments, the detecting step is performed using a biosensor. In certain embodiments, the detecting step is performed by surface plasmon resonance (SPR). In certain embodiments, the detecting step is performed by bio-layer interferometry (BLI).

In one embodiment, a method provided herein is performed in the format of an enzyme immunoassay (EIA). In another embodiment, a method provided herein is performed in the format of a radioimmunoassay (RIA). In yet another embodiment, a method provided herein is performed in the format of an enzyme linked immunosorbent assay (ELISA). In yet another embodiment, a method provided herein is performed in the format of a western blot. In yet another embodiment, a method provided herein is performed in the format of a multiplex immunoassay. In yet another embodiment, a method provided herein is performed in the format of a flow cytometric multiplex array or a bead-based multiplex array. In yet another embodiment, a method provided herein is performed in the format of an SPR immunoassay. In yet another embodiment, a method provided herein is performed in the format of a BLI immunoassay.

In one embodiment, a method provided herein comprises the steps of:
contacting a biological sample from a subject with an RXRα binder provided herein to form an RXRα binder/phosphorylated RXRα complex, wherein the RXRα binder is immobilized onto a surface of a solid phase; and
detecting the RXRα binder/phosphorylated RXRα complex;
wherein the phosphorylated RXRα comprises a phosphorylated serine at position 56 or 70.

In certain embodiments, the solid phase is a biosensor. In certain embodiments, the solid phase is an SPR biosensor, and thus the method is performed in the format of an SPR immunoassay. In certain embodiments, the solid phase is a BLI biosensor, and thus the method is performed in the format of a BLI immunoassay.

In another embodiment, a method provided herein comprises the steps of:
contacting a biological sample from a subject with an RXRα binder provided herein to form an RXRα binder/phosphorylated RXRα complex, wherein the RXRα binder is immobilized onto a surface of a solid phase;
contacting the RXRα binder/phosphorylated RXRα complex with a detection agent to form a detectable complex; and
detecting the detectable complex;
wherein the phosphorylated RXRα comprises a phosphorylated serine at position 56 or 70.

In certain embodiments, the detection agent is a detection antibody (i.e., a labelled secondary antibody) or a labelled antigen-binding fragment thereof.

In yet another embodiment, a method provided herein comprises the steps of: contacting a biological sample from a subject with an RXRα binder provided herein to form an RXRα binder/phosphorylated RXRα complex, wherein the RXRα binder is immobilized onto a surface of a solid phase;
contacting the RXRα binder/phosphorylated RXRα complex with a detection antibody to form a detectable complex; and
detecting the detectable complex;
wherein the phosphorylated RXRα comprises a phosphorylated serine at position 56 or 70.

In yet another embodiment, a method provided herein comprises the steps of:
contacting a biological sample from a subject with an RXRα binder provided herein to form an RXRα binder/phosphorylated RXRα complex, wherein the RXRα binder is immobilized onto a surface of a solid phase;
contacting the RXRα binder/phosphorylated RXRα complex with a detection antibody to form a detectable complex on the surface of the solid phase;
contacting the detectable complex with a substrate to generate a detectable signal; and detecting the detectable signal;
wherein the phosphorylated RXRα comprises a phosphorylated serine at position 56 or 70.

In yet another embodiment, a method provided herein comprises the steps of:
immobilizing an RXRα binder provided herein onto a surface of a solid phase;
blocking the surface of the solid phase to prevent nonspecific bindings;
contacting a biological sample from a subject with the immobilized RXRα binder to form an RXRα binder/phosphorylated RXRα complex on the surface of the solid phase;
contacting the RXRα binder/phosphorylated RXRα complex with a detection antibody to form a detectable immunocomplex; and
detecting the detectable complex;
wherein the phosphorylated RXRα comprises a phosphorylated serine at position 56 or 70.

In still another embodiment, a method provided herein comprises the steps of:
immobilizing an RXRα binder provided herein onto a surface of a solid phase;
blocking the surface of the solid phase to prevent nonspecific bindings;
contacting a biological sample from a subject with the immobilized RXRα binder to form an RXRα binder/phosphorylated RXRα complex on the surface of the solid phase;
contacting the RXRα binder/phosphorylated RXRα complex with a detection antibody to form a detectable complex;
contacting the detectable complex with a substrate to generate a detectable signal; and
detecting the detectable signal;
wherein the phosphorylated RXRα comprises a phosphorylated serine at position 56 or 70.

In certain embodiments, the detection antibody is an antibody or an antigen-binding fragment thereof, which is specific to the RXRα. In certain embodiments, the detection antibody does not compete with an RXRα binder provided herein for binding to a phosphopeptide of SEQ ID NO: 3.

In certain embodiments, the detection antibody is a labelled monoclonal antibody or a labelled antigen-binding fragment thereof. In certain embodiments, the detection antibody is a labelled polyclonal antibody or a labelled antigen-binding fragment thereof.

In certain embodiments, the detection antibody is a biotinylated detection antibody that specifically binds to a labeled avidin, streptavidin, or neutravidin.

In certain embodiments, the detection antibody is a chicken, donkey, goat, guinea pig, hamster, mouse, rabbit, rat, or sheep antibody.

In certain embodiments, the detection antibody comprises a reporter. In certain embodiments, the reporter is a chromogenic reporter. In certain embodiments, the chromogenic reporter is an enzyme. In certain embodiments, the chromogenic reporter is a peroxidase. In certain embodiments, the chromogenic reporter is a horseradish peroxidase or alkaline peroxidase. In certain embodiments, the chromogenic reporter is a horseradish peroxidase. In certain embodiments, the chromogenic reporter is an alkaline peroxidase.

In certain embodiments, the detection antibody is an enzyme conjugated secondary antibody. In certain embodiments, the detection antibody is a secondary antibody conjugated with a peroxidase. In certain embodiments, the detection antibody is a secondary antibody conjugated with a horseradish peroxidase or alkaline peroxidase. In certain embodiments, the detection antibody is a secondary antibody conjugated with a horseradish peroxidase. In certain embodiments, the detection antibody is a secondary antibody conjugated with an alkaline peroxidase.

In certain embodiments, the enzyme conjugated secondary antibody described herein requires a substrate to generate a signal for detection. In certain embodiments, the substrate is a colorimetric substrate. In certain embodiments, the substrate is a fluorescent substrate. In certain embodiments, the substrate is a chemiluminescent substrate. In certain embodiments, the substrate is an electrochemiluminescent substrate. Suitable substrates for a horseradish peroxidase include, but are not limited to, 2,2′-azinobis(3-ethylbenzothiazoline-6-sulfonic acid) diammonium salt (ABTS), luminol, O-phenylenediamine dihydrochloride (OPD), 3,3′,5,5′-tetramethylbenzidine (TMB), AMPLEX™ RED, AMPLEX™ ULTRARED, QUANTABLU™, QUANTARED™, and SUPERSIGNAL™. Suitable substrates for an alkaline peroxidase include, but are not limited to, adamantyl 1, 2-dioxetane aryl phosphate (AMPPD), p-nitrophenyl phosphate (PNPP), CDP-STAR™, CSPD™, and DYNALIGHT™.

In certain embodiments, the reporter is a colorimetric reporter. In certain embodiments, the reporter is a color particle. In certain embodiments, the reporter is a color microparticle or microsphere. In certain embodiments, the reporter is a color nanoparticle. In certain embodiments, the reporter is a gold particle. In certain embodiments, the reporter is a gold microparticle. In certain embodiments, the is a gold nanoparticle. In certain embodiments, the reporter is a colloidal gold nanoparticle. In certain embodiments, the reporter is a colored latex particle. In certain embodiments, the reporter is a colored latex microparticle. In certain embodiments, the reporter is a colored latex nanoparticle. In certain embodiments, the reporter is a colored polystyrene particle. In certain embodiments, the reporter is a colored polystyrene microparticle. In certain embodiments, the reporter is a colored polystyrene nanoparticle.

In certain embodiments, the reporter is a fluorescent reporter. In certain embodiments, the reporter is an organic fluorophore. In certain embodiments, the reporter is a fluorescent protein. Suitable fluorescent reporters include, but are not limited to, a cyanine dye (e.g., Cy2, Cy3, Cy3B, Cy3.5, Cy5, Cy5.5, and Cy7), a fluorescein dye (e.g., fluorescein isothiocyanate (FITC) and fluorescein diacetate), a rhodamine dye (e.g., rhodamine 6G, rhodamine 123, rhodamine B, rhodamine red, sulforhodamine B (SRB), sulforhodamine 101 (TEXAS RED™), carboxytetramethylrhodamine (TAMRA), tetramethylrhodamine (TMR), and tetramethylrhodamine isothiocyanate (TRITC)), an ALEXA FLUOR™ dye (e.g., ALEXA FLUOR™ 405, ALEXA FLUOR™ 530, ALEXA FLUOR™ 488, ALEXA FLUOR™ 500, ALEXA FLUOR™ 514, ALEXA FLUOR™ 532, ALEXA FLUOR™ 546, ALEXA FLUOR™ 555, ALEXA FLUOR™ 568, ALEXA FLUOR™ 594, ALEXA FLUOR™ 610, ALEXA FLUOR™ 633, ALEXA FLUOR™ 635, ALEXA FLUOR™ 647, ALEXA FLUOR™660, ALEXA FLUOR™680, ALEXA FLUOR™700, ALEXA FLUOR™750, and ALEXA FLUOR™ 790), a DYLIGHT™ FLUOR dye (e.g., DYLIGHT™ FLUOR 350, DYLIGHT™ FLUOR 405, DYLIGHT™ FLUOR 488, DYLIGHT™ FLUOR 550, DYLIGHT™ FLUOR 594, DYLIGHT™ FLUOR 633, DYLIGHT™ FLUOR 650, DYLIGHT™ FLUOR 680, DYLIGHT™ FLUOR 755, and DYLIGHT™ FLUOR 800), blue fluorescent protein (BFP), cyan fluorescent protein (CFP), green fluorescent protein (GFP), yellow fluorescent protein (YFP), a phycobiliprotein (e.g., allophycoerythrin, B-phycoerythrin, C-phycoerythrin, or R-phycoerythrin), and a quantum dot.

In certain embodiments, the reporter is a chemiluminescent reporter. In certain embodiment, the chemiluminescent reporter is an acridinium or ruthenium ester. In certain embodiments, the reporter is an electrochemiluminescent reporter. In certain embodiments, the electrochemiluminescent reporter is tris(2,2′-bipyridyl)ruthenium (11) chloride or dichlorotris(1,10-phenanthroline)ruthenium (II).

In certain embodiments, the reporter is a radioactive reporter. In certain embodiments, the radioactive reporter is a ³H, ¹⁵I, ³⁵S, ¹⁴C, ³²P, or ³³P containing reporter.

In certain embodiments, the RXRα binder is immobilized covalently onto a surface of a solid phase. In certain embodiments, the RXRα binder is immobilized noncovalently onto a surface of a solid phase. In certain embodiments, the RXRα binder is immobilized onto a surface of a solid phase via a specific ligand/receptor interaction. In certain embodiments, the RXRα binder is a biotinylated RXRα binder and a surface of a solid phase is surface-coated with avidin, streptavidin, or neutravidin.

In certain embodiments, the solid phase is a bead, a disc, a gel, a membrane, a sheet, a strip, or a well in a microplate.

In certain embodiments, the solid phase is a bead. In certain embodiments, the solid phase is a particle. In certain embodiments, the solid phase is a metal particle. In certain embodiments, the solid phase is a polymer bead. In certain embodiments, the solid phase is a microparticle. In certain embodiments, the solid phase is a nanoparticle. In certain embodiments, the solid phase is a microparticle, comprising copper, gold, platinum, or silver. In certain embodiments, the solid phase is a nanoparticle, comprising copper, gold, platinum, or silver. In certain embodiments, the solid phase is a carbon nanoparticle. In certain embodiments, the solid phase is a magnetic or paramagnetic bead. In certain embodiments, the solid phase is a bead comprising silica, latex, polyacrylate, polycarbonate, polyethylene, polyester, polypropylene, polystyrene, polyvinylidene difluoride (PVDF), or nylon. In certain embodiments, the solid phase is a latex bead. In certain embodiments, the solid phase is a polystyrene bead. In certain embodiments, the particle or bead comprises a reporter for detection.

In certain embodiments, the solid phase is a membrane. In certain embodiments, the solid phase is a porous membrane. In certain embodiments, the solid phase is a nitrocellulose, nylon, polyethersulfone, or PVDF membrane. In certain embodiments, the solid phase is a nitrocellulose membrane. In certain embodiments, the solid phase is a PVDF membrane. In certain embodiments, the solid phase is a sheet or strip. In certain embodiments, the solid phase is a nitrocellulose, nylon, polyethersulfone, or PVDF sheet. In certain embodiments, the solid phase is a nitrocellulose sheet. In certain embodiments, the solid phase is a PVDF sheet. In certain embodiments, the solid phase is a nitrocellulose, nylon, polyethersulfone, or PVDF strip. In certain embodiments, the solid phase is a nitrocellulose strip. In certain embodiments, the solid phase is a PVDF strip. In certain embodiments, the solid phase is a well in a microplate. In certain embodiments, the solid phase is a well in a polystyrene microplate.

In one embodiment, a method provided herein is performed in the format of a lateral flow assay.

In one embodiment, a method provided herein comprises the steps of:
contacting a biological sample from a subject with an RXRα binder provided herein to form an RXRα binder/phosphorylated RXRα complex, wherein the RXRα binder comprises a reporter;
contacting the RXRα binder/phosphorylated RXRα complex with a capture agent to capture the RXRα binder/phosphorylated RXRα complex to form a detectable complex, wherein the capture agent is immobilized onto a surface of a membrane; and
detecting the detectable complex;
wherein the phosphorylated RXRα comprises a phosphorylated serine at position 56 or 70.

In certain embodiments, the capture agent is a capture antibody or an antigen-binding fragment thereof.

In another embodiment, a method provided herein comprises the steps of: contacting a biological sample from a subject with an RXRα binder provided herein to form an RXRα binder/phosphorylated RXRα complex, wherein the RXRα binder comprises a reporter;
contacting the RXRα binder/phosphorylated RXRα complex with a capture antibody to capture the RXRα binder/phosphorylated RXRα complex to form a detectable complex, wherein the capture agent is immobilized onto a surface of a membrane; and
detecting the detectable complex;
wherein the phosphorylated RXRα comprises a phosphorylated serine at position 56 or 70.

In certain embodiments, the capture antibody is an antibody or an antigen-binding fragment thereof, which binds to the RXRα. In certain embodiments, the capture antibody does not compete with an RXRα binder provided herein for binding to a phosphopeptide of SEQ ID NO: 3.

In certain embodiments, the capture antibody is a monoclonal antibody or an antigen-binding fragment thereof. In certain embodiments, the capture antibody is a polyclonal antibody or a labelled antigen-binding fragment thereof.

In certain embodiments, the capture antibody is a biotinylated capture antibody that specifically binds to a labeled avidin, streptavidin, or neutravidin.

In certain embodiments, the capture antibody is a chicken, donkey, goat, guinea pig, hamster, mouse, rabbit, rat, or sheep antibody.

In certain embodiments, the capture antibody is immobilized covalently onto a surface of a membrane. In certain embodiments, the capture antibody is immobilized noncovalently onto a surface of a membrane. In certain embodiments, the capture antibody is immobilized onto a surface of a membrane via a specific ligand/receptor interaction. In certain embodiments, the capture antibody is a biotinylated antibody and a surface of a membrane is coated with avidin, streptavidin, or neutravidin.

In certain embodiments, the membrane is a porous membrane. In certain embodiments, the membrane is a nitrocellulose, nylon, polyethersulfones, or PVDF membrane. In certain embodiments, the membrane is a nitrocellulose membrane. In certain embodiments, the membrane is a PVDF membrane.

In one embodiment, a method provided herein is performed without a reporter. In one embodiment, a method provided herein is performed in the format of an SPR immunoassay. In another embodiment, a method provided herein is performed in the format of a BL1 immunoassay.

In one embodiment, a method provided herein comprises the steps of: contacting a biological sample from a subject with an RXRα binder provided herein immobilized onto a surface of a biosensor to generate a detectable signal; and
detecting the detectable signal;
wherein the phosphorylated RXRα comprises a phosphorylated serine at position 56 or 70.

In another embodiment, a method provided herein comprises the steps of:
immobilizing an RXRα binder provided herein onto a surface of a biosensor;
contacting a biological sample from a subject with the recombinant protein to generate a detectable signal; and
detecting the detectable signal;
wherein the phosphorylated RXRα comprises a phosphorylated serine at position 56 or 70.

In certain embodiments, the biosensor is a SPR biosensor. In certain embodiments, the biosensor is a BLI biosensor.

In certain embodiments, the RXRα binder is immobilized covalently onto a surface of a biosensor. In certain embodiments, the RXRα binder is immobilized noncovalently onto a surface of a biosensor. In certain embodiments, the RXRα binder is immobilized onto a surface of a biosensor via a specific ligand/receptor interaction. In certain embodiments, the RXRα binder is biotinylated and a surface of a biosensor is coated with avidin, streptavidin, or neutravidin.

In one embodiment, provided herein is a device for detecting a phosphorylated RXRα in a biological sample, comprising an RXRα binder provided herein, wherein the phosphorylated RXRα comprises a phosphorylated serine at position 56 or 70.

In another embodiment, provided herein is a kit for detecting a phosphorylated RXRα in a biological sample, comprising an RXRα binder provided herein, wherein the phosphorylated RXRα comprises a phosphorylated serine at position 56 or 70.

RXRα/PLK1 Modulators

In one embodiment, provided herein is a method of treating, preventing, or ameliorating one or more symptoms of a proliferative disease in a subject, comprising administering a therapeutically effective amount of an RXRα/PLK1 modulator that inhibits the interaction of a PLK1 with an RXRα comprising a phosphorylated serine at position 56 or 70.

In certain embodiments, an RXRα/PLK1 modulator provided herein is (E)-N′-((2-hydroxynaphthalen-1-yl)methylene)-2-(4-methoxyphenyl)acetohydrazide A1, or a tautomer, a mixture of two or more tautomers, or an isotopic variant thereof; or a pharmaceutically acceptable salt, solvate, hydrate, or prodrug thereof.

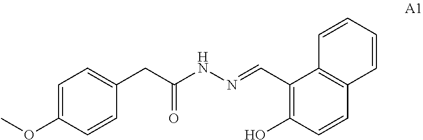 [SMILES: COc1ccc(CC(=O)N/N=C/c2c(O)ccc3ccccc23)cc1]

In certain embodiments, the RXRα comprises a phosphorylated serine at position 56. In certain embodiments, the RXRα comprises a phosphorylated serine at position 70. In certain embodiments, the RXRα comprises a phosphorylated serine at position 56 or 70 as set forth in SEQ ID NO: 1. In certain embodiments, the RXRα comprises a phosphorylated serine at position 56 as set forth in SEQ ID NO: 1. In certain embodiments, the RXRα comprises a phosphorylated serine at position 70 as set forth in SEQ ID NO: 1. In certain embodiments, the RXRα is a human RXRα. In certain embodiments, the RXRα is a human RXRα. In certain embodiments, the RXRα has an amino acid sequence of SEQ ID NO: 1.

In certain embodiments, the proliferative disease is cancer. In certain embodiments, the cancer is a solid tumor. In certain embodiments, the cancer is a hematologic malignancy.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is unresectable.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the cancer is breast cancer, cervical cancer, colorectal cancer, cutaneous squamous cell carcinoma (CSCC), endometrial carcinoma, esophageal cancer, gastric cancer, head and neck squamous cell cancer (HNSCC), hepatocellular carcinoma (HCC), Hodgkin lymphoma, melanoma. Merkel cell carcinoma (MCC), a microsatellite instability cancer, a mismatch repair deficient cancer, non-small cell lung cancer (NSCLC), primary mediastinal large B-cell lymphoma (PMBCL), renal cell carcinoma (RCC), small cell lung cancer (SCLC), or urothelial cancer (UC).

In certain embodiments, the cancer is leukemia. In certain embodiments, the cancer is acute lymphocytic leukemia (ALL), acute myeloid leukemia (AML), chronic lymphocytic leukemia (CLL), or chronic myeloid leukemia (CML). In certain embodiments, the cancer is ALL. In certain embodiments, the cancer is AML. In certain embodiments, the cancer is CLL. In certain embodiments, the cancer is CML.

In certain embodiments, the cancer is a solid tumor. In certain embodiments, the cancer is an unresectable solid tumor.

In certain embodiments, the therapeutically effective amount of an RXRα/PLK1 modulator provided herein is ranging from about 0.1 to about 100 mg/kg/day, from about 0.1 to about 50 mg/kg/day, from about 0.1 to about 60 mg/kg/day, from about 0.1 to about 50 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount of an RXRα/PLK1 modulator provided herein is ranging from about 0.1 to about 100 mg/kg/day. In another embodiment, the therapeutically effective amount of an RXRα/PLK1 modulator provided herein is ranging from about 0.1 to about 50 mg/kg/day. In yet another embodiment, the therapeutically effective amount of an RXRα/PLK1 modulator provided herein is ranging from about 0.1 to about 60 mg/kg/day. In yet another embodiment, the therapeutically effective amount of an RXRα/PLK1 modulator provided herein is ranging from about 0.1 to about 50 mg/kg/day. In yet another embodiment, the therapeutically effective amount of an RXRα/PLK1 modulator provided herein is ranging from about 0.1 to about 25 mg/kg/day. In yet another embodiment, the therapeutically effective amount of an RXRα/PLK1 modulator provided herein is ranging from about 0.1 to about 20 mg/kg/day. In yet another embodiment, the therapeutically effective amount of an RXRα/PLK1 modulator provided herein is ranging from about 0.1 to about 15 mg/kg/day. In yet another embodiment, the therapeutically effective amount of an RXRα/PLK1 modulator provided herein is ranging from about 0.1 to about 10 mg/kg/day. In still another embodiment, the therapeutically effective amount of an RXRα/PLK1 modulator provided herein is ranging from about 0.1 to about 5 mg/kg/day.

It is understood that the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both. For example, a dose of 1 mg/m²/day for a 65 kg human is approximately equal to 58 mg/kg/day.

In certain embodiments, the therapeutically effective amount of an RXRα/PLK1 modulator described herein is ranging from about 1 to about 5,000 mg per day, from about 1 to about 1,000 mg per day, from about 2 to about 500 mg per day, from about 5 to about 250 mg per day, from about 10 to about 200 mg per day, or from about 10 to about 100 mg per day. In one embodiment, the therapeutically effective amount of an RXRα/PLK1 modulator described herein is ranging from about 1 to about 5,000 mg per day. In another embodiment, the therapeutically effective amount of an RXRα/PLK1 modulator described herein is ranging from about 1 to about 1,000 mg per day. In yet another embodiment, the therapeutically effective amount of an RXRα/PLK1 modulator described herein is ranging from about 2 to about 500 mg per day. In yet another embodiment, the therapeutically effective amount of an RXRα/PLK1 modulator described herein is ranging from about 5 to about 250 mg per day. In yet another embodiment, the therapeutically effective amount of an RXRα/PLK1 modulator described herein is ranging from about 10 to about 200 mg per day. In still another embodiment, the therapeutically effective amount of an RXRα/PLK1 modulator described herein is ranging from about 10 to about 100 mg per day.

Depending on the disease to be treated and the subject's condition, an RXRα/PLK1 modulator provided herein may be administered by oral, parenteral (e.g., intramuscular, intraperitoneal, intravenous. CIV, intracisternal injection or infusion, subcutaneous injection, or implant), inhalation, nasal, vaginal, rectal, sublingual, or topical (e.g., transdermal or local) routes of administration. An RXRα/PLK1 modulator provided herein may be formulated in suitable dosage unit with a pharmaceutically acceptable excipient, carrier, adjuvant, or vehicle, appropriate for each route of administration.

In one embodiment, an RXRα/PLK1 modulator provided herein is administered orally. In another embodiment, an RXRα/PLK1 modulator provided herein is administered parenterally. In yet another embodiment, an RXRα/PLK1 modulator provided herein is administered intravenously. In yet another embodiment, an RXRα/PLK1 modulator provided herein is administered intramuscularly. In yet another embodiment, an RXRα/PLK1 modulator provided herein is administered subcutaneously. In still another embodiment, an RXRα/PLK1 modulator provided herein is administered topically.

An RXRα/PLK1 modulator provided herein can be delivered as a single dose such as, e.g., a single bolus injection, or oral tablets or pills; or over time such as, e.g., continuous infusion over time or divided bolus doses over time. An RXRα/PLK1 modulator provided herein can be administered repetitively if necessary, for example, until the subject experiences stable disease or regression, or until the subject experiences disease progression or unacceptable toxicity. Stable disease or lack thereof is determined by methods known in the art such as evaluation of subject's symptoms, physical examination, visualization of the cancer that has been imaged using X-ray, CAT, PET, or MRI scan and other commonly accepted evaluation modalities.

An RXRα/PLK1 modulator provided herein can be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of an RXRα/PLK1 modulator provided herein is administration for one to six days per week, administration in cycles (e.g., daily administration for two to eight consecutive weeks, then a rest period with no administration for up to one week), or administration on alternate days.

In certain embodiments, an RXRα/PLK1 modulator provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

An RXRα/PLK1 modulator provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of an RXRα/PLK1 modulator provided herein is independent of the route of administration of a second therapy. In one embodiment, an RXRα/PLK1 modulator provided herein is administered orally. In another embodiment, an RXRα/PLK1 modulator provided herein is administered intravenously. Thus, in accordance with these embodiments, an RXRα/PLK1 modulator provided herein is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, an RXRα/PLK1 modulator provided herein and a second therapy are administered by the same mode of administration, orally or by IV. In another embodiment, an RXRα/PLK1 modulator provided herein is administered by one mode of administration, e.g., by IV, whereas the second agent (an anticancer agent) is administered by another mode of administration, e.g., orally.

In certain embodiments, the subject is a mammal. In certain embodiments, the subject is a human.

In one embodiment, provided herein is a method of inhibiting the growth of a cell, comprising contacting the cell with an effective amount of an RXRα/PLK1 modulator that inhibits the interaction of a PLK1 with an RXRα comprising a phosphorylated serine at position 56 or 70.

In another embodiment, provided herein is a method of inducing apoptosis in a cell, comprising contacting the cell with an effective amount of an RXRα/PLK1 modulator that inhibits the interaction of a PLK1 with an RXRα comprising a phosphorylated serine at position 56 or 70.

In yet another embodiment, provided herein is a method of inhibiting mitotic progression in a cell, comprising contacting the cell with an effective amount of an RXRα/PLK1 modulator that inhibits the interaction of a PLK1 with an RXRα comprising a phosphorylated serine at position 56 or 70.

In certain embodiments, the cell is a cancerous cell. In certain embodiments, the cell is a cell of refractory and/or relapsed cancer. In certain embodiments, the cell is a cell of refractory cancer. In certain embodiments, the cell is a cell of relapsed cancer. In certain embodiments, the cell is a cell of metastatic cancer.

In certain embodiments, the cancerous cell is drug-resistant. In certain embodiment, the cancerous cell is multidrug-resistant. In certain embodiments, the cancerous cell is resistant to a chemotherapy. In certain embodiments, the cancerous cell is resistant to an immunotherapy. In certain embodiments, the cancerous cell is resistant to a standard therapy for the cancer.

In certain embodiments, the cancerous cell is a cell of breast cancer, cervical cancer, colorectal cancer, cutaneous squamous cell carcinoma (CSCC), endometrial carcinoma, esophageal cancer, gastric cancer, head and neck squamous cell cancer (HNSCC), hepatocellular carcinoma (HCC), Hodgkin lymphoma, melanoma, Merkel cell carcinoma (MCC), a microsatellite instability cancer, a mismatch repair deficient cancer, non-small cell lung cancer (NSCLC), primary mediastinal large B-cell lymphoma (PMBCL), renal cell carcinoma (RCC), small cell lung cancer (SCLC), or urothelial cancer (UC).

In certain embodiments, the cancerous cell is a leukemia cell. In certain embodiments, the cancerous cell is a cell of acute lymphocytic leukemia (ALL), acute myeloid leukemia (AML), chronic lymphocytic leukemia (CLL), or chronic myeloid leukemia (CML). In certain embodiments, the cancerous cell is an ALL cell. In certain embodiments, the cancerous cell is an AML cell. In certain embodiments, the cancerous cell is a CLL cell. In certain embodiments, the cancerous cell is a CML cell.

In certain embodiments, the cancerous cell is a cell of a solid tumor.

The disclosure will be further understood by the following non-limiting examples.

### EXAMPLES

### General Procedures

Cell Lines

HeLa human cervical carcinoma cells were maintained in the HYCLONE™ MEM/EBSS medium containing 10% fetal bovine serum (FBS). A549 human lung adenocarcinoma cells were maintained in the Kaighn's Modification of the Ham's F-12 medium containing 10% FBS. HepG2 human hepatocellular carcinoma cells, SMMC-7721 human hepatocellular carcinoma cells, SK-Hep-1 human hepatocellular carcinoma cells, Bel-7402 human hepatocellular carcinoma cells, MCF-7 human breast cancer cells, BxPC-3 human pancreas cancer cells, SW480 human colon cancer cells, mouse embryonic fibroblasts (MEF), mouse melanoma B16F10 cells, and mouse breast cancer 4T1 cells were cultured in a DMEM medium containing 10% FBS. Normal liver cells (QSG-7701) were cultured in a RPMI-1640 medium supplemented with 10% FBS. THLE-2 cells derived from primary normal liver cells were cultured in a bronchial epithelial growth medium (BEGM) supplemented with the BULLET KIT™, of which the gentamycin/amphotericin (GA) and epinephrine were discarded and extra 5 ng/mL EGF, 70 ng/mL phosphoethanolamine, and 10% FBS were added.

Mice

BALB/C female nude mice (4-6 weeks old) and C57BL/6 male mice (6-8 weeks old) were maintained in animal room with 12 hr light/12 hr dark cycles; and used in the experiments described herein.

Transfection

Transient transfection was performed using LIPOFECTAMINE™ 2000. To establish stable clones expressing RXRα or RXR-S56A/S70A(2A), p3×FLAG-CMV-10-RXRα, p3×FLAG-CMV-10-RXR-2A, or empty vector p 3×FLAG-CMV-10 plasmids were transfected into cells using LIPOFECTAMINE™ 2000. After 48-hr transfection, media were each replaced with a G418-containing (1 mg/mL) medium. Individual colonies were picked up after a 10-day selection. Transfection efficiency was determined by examining the expression of RXRα. Stably transfected cells were maintained in a medium containing 200 μg/mL G418.

Cell Synchronization

Cells were synchronized at GUS boundary by a double thymidine block. Briefly, cells cultured in a medium containing 2 mM thymidine for 16 hr were released into a normal medium for 8 hr, subjected to a second thymidine block for 16 hr, and then released into a fresh medium again. Alternatively, cells were synchronized at prometaphase by a thymidine-nocodazole arrest (an 18-hr thymidine arrest and a 5-hr release, followed by a 5-hr 100 ng/mL nocodazole arrest).

Centrosome Isolation

Centrosomes from HeLa cells were isolated by discontinuous gradient ultracentrifugation as described (Wigley et al., J. Cell. Biol. 1999, 145, 481-90). Briefly, HeLa cells released from a double thymidine block for 10 hr were treated with 1 mg/mL cytochalasin D and 0.2 mM nocodazole for 1 hr at 37° C. before harvesting. The cells were collected by trypsinization and centrifugation, and the resulting pellet was washed in PBS, followed by 0.1×PBS/8% sucrose. The cells were resuspended in 2 mL of 0.1×TBS/8% sucrose, followed by addition of 8 mL of a lysis buffer (1 mM HEPES, pH 7.2, 0.5% NP-40, 0.5 mM MgCl₂, 0.1% β-mercaptoethanol, 1 mg/mL leupeptin, 1 mg/mL pepstatin, 1 mg/mL aprotinin, and 1 mM PMSF). The suspension was gently shaken and passed five times through a 10-mL narrow-mouth serological pipette to lyse the cells. Swollen nuclei, chromatin aggregates, and unlysed cells were removed by centrifugation at 2,500 g for 10 min. The lysis supernatant was filtered through a nylon mesh into a 250 mL centrifugation bottle. A concentrated solution of 1 M HEPES was added to obtain a final concentration of 10 mM. DNAse I was added to a final concentration of 1 μg/mL. The lysis supernatant was mixed well to form a centrosomal suspension, which was kept at 4° C. for 30 min. The mixture was gently underlaid with 1 mL of 60% sucrose solution (10 mM PIPES, pH 7.2, 0.1% Triton X-100, and 0.1% β-mercaptoethanol containing 60% by weight sucrose) and spun at 10,000 g for 30 min to sediment centrosomes onto a cushion. The upper 8 mL of the supernatant was removed and the remainder, including the cushion, containing the concentrated centrosomes, was gently vortexed and loaded onto a discontinuous sucrose gradient consisting of 70, 50, and 40% solutions from the bottom, respectively, and spun at 25,600 g for 1 hr. Fractions were collected and subjected to western blot analysis.

Fluorescence-Activated Cell Sorter (FACS) Analysis

Cells were harvested by trypsin digestion, washed with PBS, and fixed with ice-cold 70% ethanol overnight at 4° C. The fixed cells were then washed twice in PBS and treated for 10 min at room temperature with 0.5 μg/mL 4-6-diamidino-2-phenylindole (DAPI) in PBS and analyzed on a FACSCAN™ flow cytometer. Flow cytometry data were analyzed using CYTEXPERT.

RXRα Knocking Down and Knocking Out

a. CRISPR/Cas9 Genome Editing

To establish stable clones lacking RXRα, pX330-U6-Chimeric_BB-CBh-hSpCas9-RXRα (sequence: GGCGGGCCCATGCCGTTGAT; designed by CRISPR design tool) or empty control vector pX330-U6-Chimeric_BB-CBh-hSpCas9 was transfected into A549 cells by LIPOFECTAMINE™ 2000. To identify positive clones, pEGFP-C1 plasmid was transfected together with pX330 that contains a neomycin resistance cassette. Transfected cells were diluted and grown in a medium containing 800 μg/mL G418 for 14 days for selection, b. RXRα siRNA approach

To knock down RXRα, cells were transfected with siRNA duplexes using LIPOFECTAMINE™ 2000 in a serum-free tissue culture medium. Four hours after the transfection, the cells were fed with a normal medium. The cells were collected 48 hr after transfection and analyzed by western blotting, flow cytometry, and immunofluorescence staining. For siRNA rescue assays, site-directed mutagenesis was used to introduce four silent mutations into the coding region of human RXRα (nucleotides 1237-1255) cognate to the RXRα siRNA (GGAAGGUUCGCUAAGCUCU; mutations italicized), and introduction of these mutations was confirmed by sequencing.

SILAC-Based Immunoprecipitation Quantitative Proteomics

For stable isotope labeling with amino acids in cell culture (SILAC) (Ong and Mann, Nat. Protoc. 2006, 1, 2650-60), HeLa cells stably transfected with a FLAG-RXRα plasmid were grown in a SILAC DMEM “heavy” medium without lysine and arginine, supplemented with 10% dialyzed fetal calf serum, 100 units/mL penicillin, 100 units/mL streptomycin, 200 μg/mL L-proline, 100 μg/mL L-arginine HCl (13C6; 15N4), and 200 μg/mL L-lysine-2HCl (13C6; 15N2). HeLa cells stably transfected with empty control vector p3×FLAG-CMV-10 plasmid were grown in a SILAC DMEM “light” medium without lysine and arginine, supplemented with 10% dialyzed fetal calf serum, 100 units/mL penicillin, 100 units/mL streptomycin, 100 μg/mL L-arginine and 200 μg/mL L-lysine. Two cell populations were each grown in the corresponding culture medium for at least seven cell divisions by changing the medium every 2 days.

To conduct immunoprecipitation coupled with liquid chromatography tandem mass spectrometry (LC-MS/MS), two different groups of HeLa cells (light and heavy) described above were harvested and lysed in an immunoprecipitation (IP) lysis buffer (50 mM Tris-HCl, pH 7.4, 150 mM NaCl, 0.5% NP-40, 10 mM EDTA) supplemented with protease and phosphatase inhibitors. Equal amounts of cell lysates from both groups were incubated with anti-FLAG M2 beads for 2 hr at 4° C. After washed by the IP lysis buffer for three times, two samples were mixed with 1:1 and separated by 4-20% gradient SDS-PAGE. After silver staining, each gel lane was cut horizontally into 20 gel pieces, which were then in-gel destained, reduced, alkylated, and digested with trypsin at 37° C. overnight. Peptides were extracted and concentrated by centrifugation. Peptides were desalted, filtered through a C18 ziptip, and redissolved with 0.1% formic acid in ultrapure water before being analyzed by HPLC coupled with a Q-EXACTIVE™ mass spectrometer. At the end, the peptide mixtures were eluted with a gradient buffer solution (buffer A, 0.1% formic acid; buffer B, 0.1% formic acid in ACN) and separated on ACCLAIM® PEPMAP™ 100 NANOVIPAR C18 column (50 μm×15 cm, 2 μm, 100 Å) over 120 min. The eluate was then analyzed in the Q-EXACTIVE™ mass spectrometer operated in a data dependent mode. Protein identification and quantitation were automatically performed by a THERMO PROTEOME DISCOVERER™ software against the UNIPROT human protein database release 2014_08. Precursor ion mass tolerance was 10 ppm; and fragment ion mass tolerance was 0.5 Da. The FDR of protein, peptide and site was 0.01. The normalized ratio of the heavy versus light SILAC label was automatically calculated by a PD program.

Confocal Microscopy

Cells mounted on glass slides were fixed with methanol at −20° C. for 10 min, then permeabilized with 0.05% Triton X-100 in a PBS buffer for 8 min at 4° C., and blocked with 1% bovine serum in PBS for 30 min at room temperature, followed by incubation with primary antibodies at room temperature for 3 hr and detected by FITC-labeled anti-IgG (1:200), anti-goat IgG conjugated with Cy3 (1:200) at room temperature for 1 hr. The cells were costained with 40,6-diamidino-2-phenylindole (DAPI) (1:10,000 dilution) to visualize nuclei. The images were taken under a LEICA TCS SP8 confocal laser scanning microscope system or an LSM-510 confocal laser scanning microscope system.

In Situ Proximity Ligation Assay

An in situ proximity ligation assay (PLA) was performed in HeLa cells and HepG2 by using a DOULINK™ assay kit. Briefly, cells mounted on glass slides were fixed with methanol at −20° C. for 5 min, then permeabilized with 0.05% Triton X-100 in a PBS buffer for 8 min at 4° C., and blocked with a blocking solution for 60 min at 37° C., followed by incubation with primary antibodies for 60 min at 37° C. The slides were then washed three times with wash buffer A, and PLA labeled secondary antibodies (anti-mouse, minus; anti-rabbit, plus) were added and incubated for 60 min at 37° C. The slides were washed two times with wash buffer A and a ligation solution including a ligase was added for 30 min at 37° C. After ligation, the slides were washed two times with wash buffer A and followed by incubation with an amplification buffer including a polymerase for 100 min at 37° C. After amplification, the slides were washed two times with 1×wash buffer B and one time in 0.01×wash buffer B. Finally, the slides were mounted using a minimal volume of a mounting medium with DAPI and analyzed in a confocal microscope after 15 min.

Co-Immunoprecipitation

Briefly, cells were harvested in a lysis buffer (10 mM Tris, pH 7.4, 150 mM NaCl, 0.5% NP40, 5 mM ethylene diamine tetraacetic acid, containing protease inhibitors). Cell lysates were incubated with an antibody (1 μg) at 4° C. for 2 hr. Immunocomplexes were then precipitated with 30 μL of protein G-Sepharose beads. After an extensive washing with the lysis buffer, the beads were boiled in a sodium dodecyl sulfate (SDS) sample loading buffer and assessed by western blotting.

Western Blotting

Cell lysates were boiled in an SDS sample loading buffer, resolved by 10% SDS-polyacrylamide gel electrophoresis (SDS-PAGE), and transferred to PVDF membranes. The membranes were blocked with 5% milk in a Tris buffered saline and TWEEN 20 (TBST)(10 mM Tris-HCl, pH 8.0, 150 mM NaCl, 0.05% TWEEN 20) for 1 hr at room temperature. After washing twice with TBST, the membranes were incubated with primary antibodies in TBST for 1 hr and then washed twice, probed with a horseradish peroxide-linked anti-immunoglobulin (1:5,000 dilution) for 1 hr at room temperature. After three washes with TBST, immunoreactive products were visualized using enhanced chemiluminescence reagents and autoradiography.

Phosphatase and Glycosidase Assays

To determine the nature of RXRα modification during mitosis, lysates from cells released from synchronization by a double thymidine block for 10 hr were incubated with a thermosensitive alkaline phosphatase (TAP), O-glycosidase, or PNGase F at 37° C. After incubation, reactions were boiled in an SDS sample loading buffer, loaded onto a denaturating gel, and analyzed by western blotting.

Protein Purification

The plasmids (pGEX-4T1-GST-RXRα, pGEX-4T1-GST-RXRα-2A, pET28a-6×His-PLK1, and pET45a-6×His-Auror A) were expressed in Escherichia Coli BL21 strain. Cells were grown in LB broth under antibiotic selection at 37° C. until OD600 at 0.6-0.8 and protein expression was induced with 1 mM IPTG at 16° C. for 16 hr. Cells were lysed by sonication in buffer A (20 mM Tris-HCl, pH 8.0, 100 mM NaCl, 1 mM EDTA, 1% Triton X-100; for GST-RXRα protein), or buffer B (20 mM Tris-HCl, pH 8.0, 300 mM NaCl, 1% Triton X-100, 20 mM imidazole; for 6×His-PLK1 and 6×His-Aurora A proteins). Lysates were clarified by centrifugation and incubated with glutathione SEPHAROSE 4B or Ni-NTA resins in a lysis buffer. The resins were washed with the lysis buffer and eluted with 20 mM glutathione or 250 mM imidazole. The proteins were concentrated to about 2 mg/mL.

In Vitro Kinase Assays

To test whether RXRα was phosphorylated by FLAG-Cdk1/Myc-cyclin B1, 0.5-1 g purified GST-RXRα or GST-RXRα-2A was incubated with FLAG-Cdk1/Myc-cyclin B1 immunoprecipitated from mitotic HeLa cells in a kinase reaction buffer (50 mM Tris-HCl, pH 8.0, 150 mM NaCl, 100 mM DTT, 10 mM MgCl₂, 0.3 mM ATP) for 30 min at 30° C., and then analyzed by western blot. For control, mitotic cells were subjected to similar immunoprecipitation by control IgG. To test the effect of Cdk1-phosphorylated RXRα on PLK1 phosphorylation by Aurora A, GST-RXRα or GST-RXRα-2A was first subjected to a kinase reaction with FLAG-Cdk1/Myc-cyclin B1 immunoprecipitated from mitotic HeLa cells. FLAG-Cdk1/Myc-cyclin B1 were removed by centrifugation. The supernatant of the reaction, which contains phosphorylated RXRα or unphosphorylated RXRα-2A, was incubated with purified 6×His-Aurora A and 6×His-PLK1 at 30° C. for 1 hr in the presence of ATP, and analyzed by western blot. For control, immunoprecipitates using control IgG from mitotic HeLa cells were used to phosphorylate GST-RXRα, and the resulting supernatant was used.

In vitro kinase assay also performed with standard [γ-³²P]ATP labeling. Briefly, FLAG-Cdk1/Myc-cyclin B1 or FLAG-PLK1 immunoprecipitated from mitotic HeLa cells was incubated with 1 μg GST-RXRα or 1 μg Casein in a kinase buffer containing 300 μM ATP and 10 μCi [γ-³²P]ATP (3,000 Ci/mmol) at 30° C. for 45 min, and the reaction mixture was then separated by SDS/PAGE and detected by autoradiography.

Identification of Phosphorylation Sites by Mass Spectrometry

HeLa cells were transfected with constructs encoding FLAG-tagged RXRα and synchronized to the GUS boundary by a double-thymidine treatment, harvested at 10 hr after release, and subjected to immunoprecipitation using anti-FLAG antibodies. Immunoprecipitated RXRα were separated by SDS-PAGE. After silver staining, phosphorylated RXRα was cut horizontally, which was then in-gel destained, reduced, alkylated, and digested with chymotrypsin at 37° C. overnight. The sample was analyzed as described herein.

Docking Experiments

Schrodinger's GLIDE, a grid-based docking program, was used for docking studies of two peptides—ISTLSSP and ISLS(p-S)P to PLK-1 PBD. The crystal structure of PLK-1-PBD in complex with hydrocinnamoyl-derivatized PLHSpTA peptide (Protein Data Bank Code 4E67) was used. Docking was performed with the implemented standard routine in GLIDE. Conformation search was employed to generate different poses of two peptides, and then all the poses were pooled to GILDE docking, every pose kept three output conformations. The GLIDE GSCORE was used as a docking score to rank the docking results. Poses were further visually investigated to check for their interactions with the protein in the docking site. Schrodinger's MAESTRO was used as primary graphical user interfaces for the visualization of crystal structures and docking results.

Generation of Anti-pS56-RXRα Antibody

A rabbit polyclonal phosphospecific antibody (anti-pS56-RXRα) was generated using an RXRα phosphopeptide, SPISTLS(pS)PING (SEQ ID NO: 3), derived from amino acid residues 49 to 60 of RXRα. Antibodies were affinity purified using phosphorylated peptide-conjugated gels.

Partial Hepatectomy

With mice under isoflurane anesthesia and sterile conditions, two-thirds of the liver was surgically removed. Briefly, C57BL/6 male mice (8 weeks old) were placed into a plexiglass chamber for induction of anesthesia with 2% isoflurane and 2 liter/min oxygen flow. After anesthetization, the mice were maintained under anesthesia by isoflurane inhalation through a suitable mouthpiece. The left and median lobes of the liver were pushed out from an incision (about 3 cm) just under the xiphoid process and were removed with a ligature. Following surgery, the mice were returned to their cages and given free access to food and water. Mice as a control group were operated just as above except that no lobes were the lobes. Livers were fixed in a 4% neutral buffered formalin phosphate (pH 7.0) for periods not exceeding 24 hr and were subsequently embedded in paraffin. They were sliced into 4-μm sections for H&E (hematoxylin and eosin) staining and immunofluorescence. To examine hepatocytes, H&E stained-liver sections were analyzed with an image analysis system.

HCC Tumorigenesis Model

Mice (15 days old) were intraperitoneally injected with diethylnitrosamine (DEN, dissolved in PBS, 25 mg/kg) and 6 weeks later injected with carbon tetrachloride (CCl₄, dissolved in corn oil, 0.5 mL/kg) twice a week for 17 weeks. The other diet-induced mouse model of hepatocellular carcinoma was carried out as described (Clapper et al., Am. J. Physiol. Gastrointest. Liver Physiol. 2013, 305. G483-95. In brief, male C57BL/J6J mice (8 weeks old) were randomly fed with either normal chow or a diet (HFHC) enriched in fat (40% kcal, PRIMEX partially hydrogenated vegetable oil shortening), fructose (22% by weight), and cholesterol (2% by weight). After 32 weeks, mice were euthanized and livers were collected for isolation primary cells or western blot analysis.

HepG2 Xenografts

BALB/c nude mice (7-8 weeks old) were injected subcutaneously with 100 μL HepG2 cells (2×10⁶). For treatment, mice were administered with compound A1 (80 mg/kg) diluted in corn oil once every two days. Body weights and tumor sizes were measured every 2 days with tumor volumes (V) calculated by the formula of (width)²×length/2. The mice were sacrificed after 14 days of treatment and tumors were removed for additional assessments. Isolation and culture of mouse primary liver tumor cells

Briefly, a liver from a mouse was removed and washed in 1×PBS. The tumor tissue was resected from the liver and transferred to 5 mL PBS/2% FBS. The tumor tissue was minced with fine sterile scissors into fragments, followed by addition of 5 mL pre-warmed 2 mg/mL collagenase type IV/dispase. After incubated for 30 min at 37° C., the tumor cells were filtered through a 70 μm cell strainer and spined at 1,000 rpm for 5 min at 4° C. The pellet containing the liver tumor cells was then resuspended in 5 mL of 1×RBC lysis buffer and incubated on ice for 5 min. The tumor cells were spined and washed by 1×PBS/2% FBS for 2-3 times. Finally, the tumor cells were resuspended with DMEM (10% FBS, 40 ng/mL EGF, 0.008 ng/mL IGF-II) and plated in culture dishes that were pre-coated with collagen.

Mouse Hepatocyte Isolation and Culture

Mouse primary hepatocytes were isolated by a two-step liver perfusion method. Briefly, a mouse was anesthetized by a pentobarbital sodium solution (50 mg/kg body weight) intraperitoneally. The abdomen was then cut open, and the portal vein (PV) was catheterized. The liver was first perfused in situ with D-Hank's buffered solution (containing 0.5 mM EGTA) pre-warmed to 37° C. for 8-10 min with the inferior vena cava (IVC) cut for drainage and then perfused for 5 min with 100 units CDU/mL collagenase Type IV perfusate (containing 2 mM Ca2+). The livers were extirpated and placed into plates filled with DMEM at 4° C. The livers were torn apart and gently shaken to free residual cells. The cell suspension was collected and filtered through a 70-micron membrane, resuspended in a PERCOLL/DMEM/PBS (1:1:0.3) mixture and centrifuged at 50 g for 15 min at room temperature. Cell viability was examined by the trypan blue exclusion test (generally >90%). Purified hepatocytes were washed twice with DMEM, resuspended in a culture media (DMEM with 10% FBS), and then plated in culture dishes that were pre-coated with collagen.

Isolation and Culture of Primary Human Hepatic Carcinoma Cells

Tissue samples were obtained by surgical resection and processed within 2 hr. Chen et al., Oncotarget 2016, 7, 17047-59. Samples were washed with 1×SC-2 solution, minced with fine sterile scissors and scalpel into fragments of ˜1 mm³. They were then treated with pre-warmed collagenase type IV for 30 min at 37° C. and filtered through a 70 μm cell strainer. The pellet containing the liver cells was resuspended in ice cold SC-2 solution and triple centrifugation at 1,000 g for 10 min at 4° C. was performed in order to separate the purified hepatocyte population (pellet) from non-parenchymal cells (supernatant). The pellet contained the purified hepatocytes. The resolved single-cell suspensions were then re-suspended in a primary cell culture medium (DMEM, 20% FBS, 2 mM glutamine, 1 mM pyruvate, 10 mM HEPES, 100 units/mL penicillin/streptomycin, 0.1 mg/mL gentamicin, and 2 g/L fungizone), plated in 6-well plates coated with 5 μg/cm² rat tail collagen type I and maintained in culture at 37° C. and 5% CO₂.

Mouse Tissue Process and Histological Examination

Livers or transplanted tumor from mouse were fixed in 4% neutral buffered formalin phosphate (pH 7.0) for periods not exceeding 24 hr and were subsequently embedded in paraffin. They were sliced into 4-μm sections for H&E (hematoxylin and eosin) staining, immunofluorescence and for immunohistochemistry. To examine hepatocytes, H&E stained-liver sections were analyzed with an image analysis system. For immunostaining, liver sections or tumor sections were incubated with an anti-γ-tubulin (1:200 dilution), anti-pS10-H3 (1:200 dilution), anti-γ-H2AX (1:200 dilution), or cleaved-caspase-3 (1:100 dilution) antibody. Positive cells or areas were counted and measured, respectively, for at least 10 fields.

Clinical Tumor Samples

Human liver cancer tissues or colorectal cancer tissues, and their corresponding tumor adjacent normal tissues were obtained from Zhongshan Hospital Affiliated to Xiamen University and the affiliated hospital of Xuzhou Medical University. All patients have been operated in the surgical unit of the hospitals and the protocol has been approved by the Ethical and Scientific Committee of the Hospitals.

Surface Plasmon Resonance Assay

A surface plasmon resonance (SPR) assay was used to screen over 50 compounds derived from Chen et al. (ACS Med. Chem. Lett. 2014, 5, 736-41) for their binding to a purified ligand-binding domain (LBD) of RXRα (RXRα-LBD) protein by BIACORE™ T200 as described (Zeng et al., Cancer Res. 2015, 75, 2049-60; Hu et al., Mol. Cell 2017, 66, 141-53). The identified compound A1 was tested again to confirm its binding with a gradient concentrations of 0.625, 1.25, 1.56, 2.5, and 3.125 μM injected through flow cells immobilized with RXRα-LBD protein.

Statistical Analyses

All statistical analyses were performed using Prism 5 (GRAPHPAD™) and data are presented as the mean ±SEM. Unless otherwise indicated, statistical comparisons were made using student's t-test. Survival data was analyzed using the Kaplan-Meier statistical method. P<0.05 was considered statistically significant (*), P<0.01 as highly significant (**), P<0.001 as extremely significant (***), and ns as not significant.

### Example 1

### Identifying PLK1 as a Unique RXRα-Interacting Protein

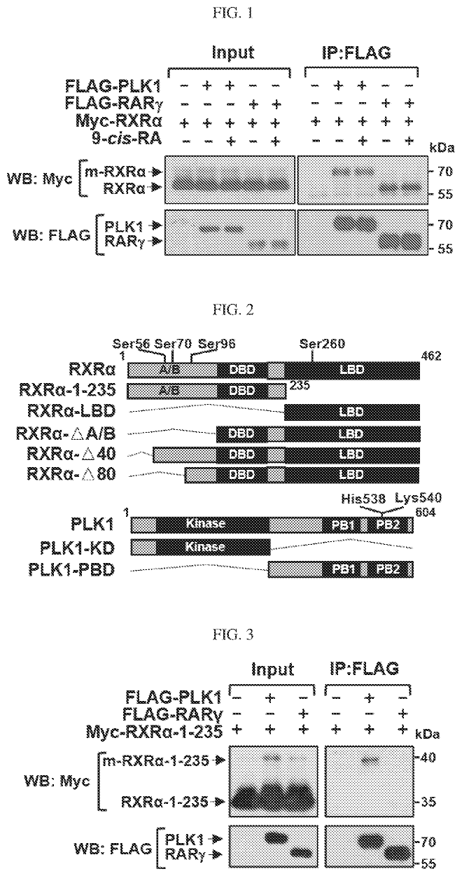

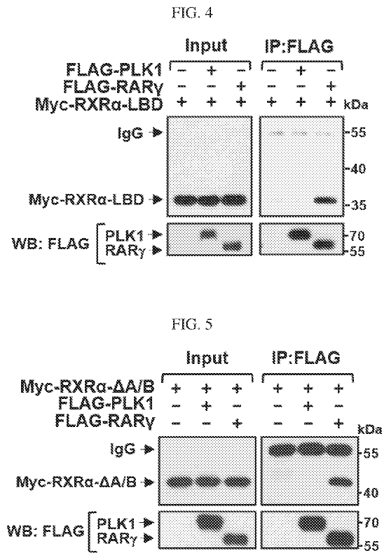

Stable isotope labeling with amino acids in cell culture (SILAC)-immunoprecipitation quantitative proteomics was used to identify new RXRα-binding proteins. A shown in FIG. 1, PLK1 strongly interacted with RXRα. Surprisingly, PLK1 interacted only with a modified form of RXRα (m-RXRα) with reduced mobility on SDS-PAGE, but not the regular 55 kDa RXRα protein. For comparison, retinoic acid receptor-γ (RARγ) interacted with the regular, but not m-RXRα. The RXRα ligand 9-cis-RA showed no apparent effect on m-RXRα interaction with PLK1. Analysis of RXRα and PLK1 mutants (FIG. 2) showed that the N-terminal portion of RXRα (RXRα-1-235) including its A/B domain and the DBD also exhibited two protein products, in which the modified one with mobility shift interacted with PLK1, but not RARγ that is known to heterodimerize with RXRα through their C-terminal LBDs (FIG. 3). Zhang et al., Nature 1992, 355, 441-6. Conversely, the LBD of RXRα expressed only as one band, which interacted with RARγ but not PLK1 (FIG. 4). RXRα mutant (RXRα-AA/B) lacking the N-terminal A/B domain failed to bind PLK1 despite its ability to interact with RARγ (FIG. 5). Thus, the A/B domain of RXRα was identified to be responsible for PLK1 binding upon appropriate modifications. Analysis of PLK1 mutants revealed that the C-terminal polo-box domain (PBD) of PLK1, a conserved phosphopeptide-binding domain that recognizes phosphorylated threonine or serine residues of partner proteins (Cheng et al., EMBO J. 2003, 22, 5757-68; Elia et al., Science 2003, 299, 1228-31; Elia et al., Cell 2003, 15, 83-95), but not its N-terminal kinase domain (KD) interacted with m-RXRα. The interaction of the PBD with RXRα was much stronger than full-length PLK1 (FIG. 6), likely due to an intramolecular interaction between KD and PBD in PLK1, which restrains the binding activity of PBD (Elia et al., Cell 2003, 115, 83-95).

### Example 2

### RXRα Modification During Mitosis

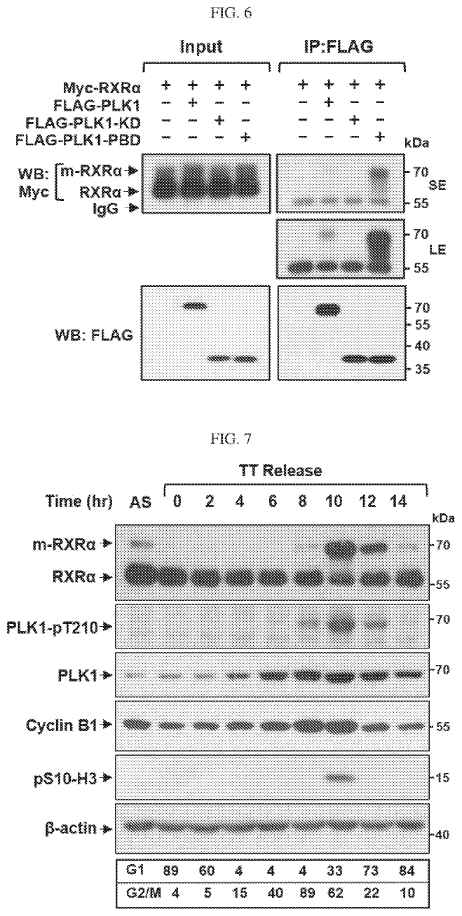

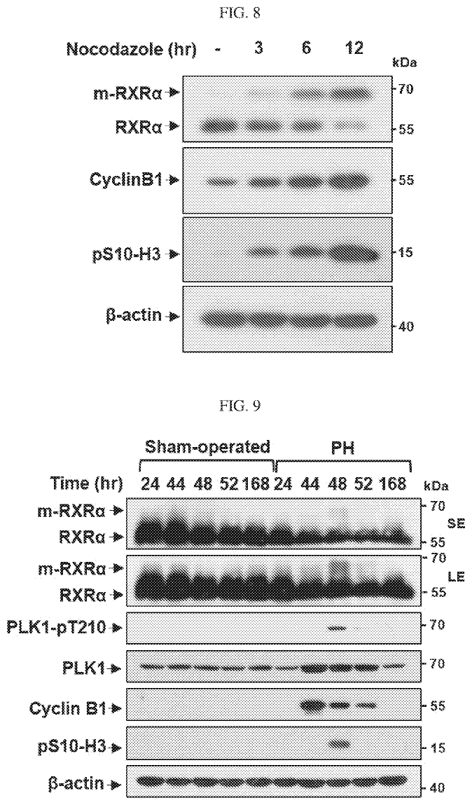

HeLa cells synchronized at GUS phase by a double-thymidine (TT) block were released into the cell cycle progressively. Immunostaining revealed that m-RXRα was clearly detected in mitotic cells released from the TT block for 10 hr (FIG. 7). As shown in FIG. 8, modification of RXRα during mitosis was also illustrated by using the microtubule depolymerizing agent nocodazole known to arrest cells at the G2/M phase. The level of m-RXRα peaked during mitosis (10 hr) when Ser 10 of histone H3 (S10-H3) was phosphorylated, an event indicative of chromosome condensation and segregation, and correlated very well with that of cyclin B1, a key component required for the activation of Cdk1. Nigg, Nat. Rev. Mol. Cell Biol. 2001, 2, 21-32; Malumbres and Barbacid, Biochem. Biophys. Res. Commun. 2009, 225, 946-51. Similarly coordinated expression of m-RXRα and cyclin BI during mitotic progression was observed in all cancer cell lines examined. Thus, RXRα undergoes specific modification during mitosis.

RXRα is highly expressed in the liver and plays a prominent role in hepatic growth, regeneration, and homeostatic functions. Bushue and Wan, J. Exp. Clin. Med. 2009, 1, 23-30. Therefore, RXRα was studied to determine whether it is modified during liver regeneration after partial hepatectomy (PH), in which most of the hepatocytes re-enter the cell cycle synchronously. Michalopoulos and DeFrances, Science 1997, 276, 60-6. Analysis of liver extracts prepared from mice subjected to PH revealed the appearance of m-RXRα around 48 hr after PH (FIG. 9), when mitotic hepatocytes were detected by H&E and immunostaining. The timing of the appearance/disappearance of m-RXRα correlated with the induction/degradation of the mitotic marker pS10-H3 and cyclin B1, demonstrating that RXRα is modified during liver regeneration.

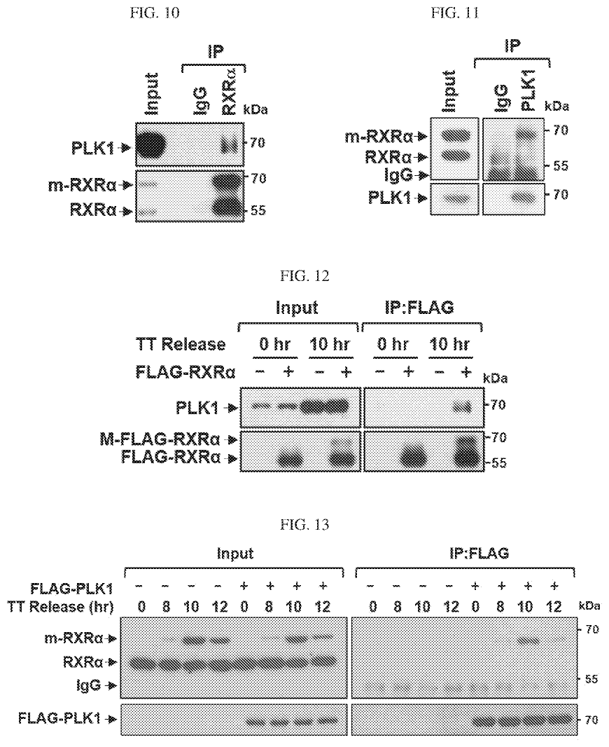

It was next determined whether mitosis-specific modification of RXRα conferred its ability to interact with PLK1. While immunoprecipitation of RXRα and m-RXRα in mitotic cells resulted in co-immunoprecipitation of PLK1 (FIG. 10), immunoprecipitation of PLK1 led to co-immunoprecipitation of only m-RXRα (FIG. 11), revealing a selective interaction of endogenous PLK1 with m-RXRα. Similar to endogenous RXRα, transfected Flag-RXRα was modified and then interacted with PLK1 in mitotic cells (FIG. 12). Transfected Flag-PLK1 also strongly interacted with modified but not regular RXRα (FIG. 13). Thus, the mitosis-specific modification of RXRα confers its ability to interact with PLK1.

### Example 3

### RXRα Phosphorylation by Cdk1 During Mitosis

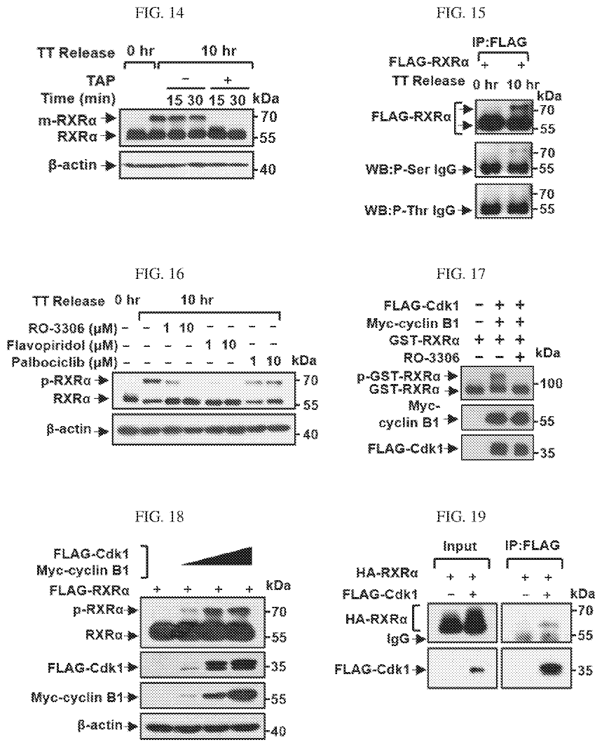

Several inhibitors and mutagenesis approaches were employed to study the nature of RXRα modification. It was determined that RXRα modification is not due to ubiquitination, sumolyation, or glycosylation. However, treatment of lysates or RXRα immunoprecipitated from mitotic cells with a thermosensitive alkaline phosphatase (TAP) resulted in disappearance of modified but not regular RXRα (FIG. 14). The modified FLAG-RXRα reacted to anti-pSer but not anti-pThr antibody (FIG. 15). Thus, Ser-mediated phosphorylation of RXRα (p-RXRα; p-RXRα refers to m-RXRα hereafter) is responsible for its mobility shift on SDS/PAGE.

To identify the kinase responsible for RXRα phosphorylation, it was first determined whether c-Jun N-terminal kinase (JNK) known to phosphorylate RXRα (Adam-Stitah et al., J. Biol. Chem. 1999, 274, 18932-41) is involved. Treatment of mitotic cells with the JNK inhibitor SP600125 had no effect on RXRα shift. It was also determined whether PLK1 is responsible for RXRα shift because the timing of RXRα shift coincided with PLK1 activation (FIGS. 7 and 9). Knocking down PLK1 or treating cells with the PLK1 inhibitor BI2536 did not inhibit RXRα shift. Inactivating Aurora A, which activates PLK1 (Macurek et al., Nature 2008, 455, 119-23; Joukov and De Nicolo, Sci. Signal. 2018, 11, eaar4195), by the Aurora A inhibitor VX680 attenuated PLK1 activation but not RXRα shift. Thus, JNK and PLK1 are not responsible for RXRα modification. Given the facts that the PBD of PLK1, which often docks to consensus sites for Cdk1 phosphorylation (Cheng et al., EMBO J. 2003, 22, 5757-68; Elia et al., Science 2003, 299, 1228-31; Elia et al., Cell 2003, 115, 83-95), interacts with p-RXRα (FIG. 6) and that Cdk1 activation correlates well with RXRα shift during mitotic progression (FIGS. 7 to 9), Cdk1 was studied to determine whether it is responsible for RXRα phosphorylation. Cdk1-selective inhibitor RO-3306 and nonselective inhibitor flavopiridol strongly inhibited RXRα shift in a dose dependent manner, while palbociclib, a Cdk4/6 inhibitor, had no effect (FIG. 16). Purified GST-RXRα protein was shifted upon incubation with FLAG-Cdk1 and myc-cyclin BI immunoprecipitated from mitotic HeLa cells (FIG. 17), whereas transfected Flag-RXRα was also shifted upon cotransfection with Cdk1 and Cyclin B1 (FIG. 18). RXRα could also interact with Cdk1 (FIG. 19). In vitro kinase assay with [γ-³²P]ATP labeling revealed that Cdk1 phosphorylated RXRα and caused its mobility shift. Interestingly, PLK1 also phosphorylated RXRα, which, however, did not result in its mobility shift. Together, these results identified Cdk1 as the kinase responsible for RXRα phosphorylation during mitosis.

### Example 4

### Determining Cdk1 Phosphorylation Site in RXRα and its Role in PLK1 Interaction

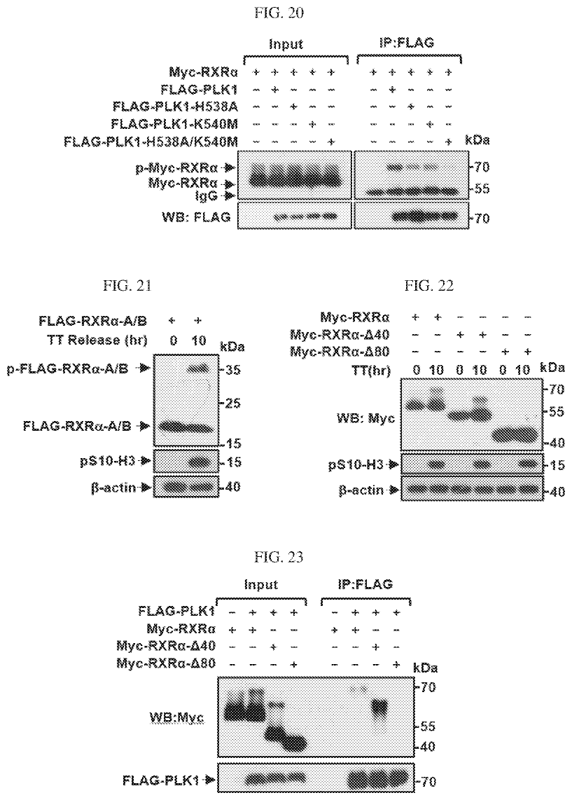

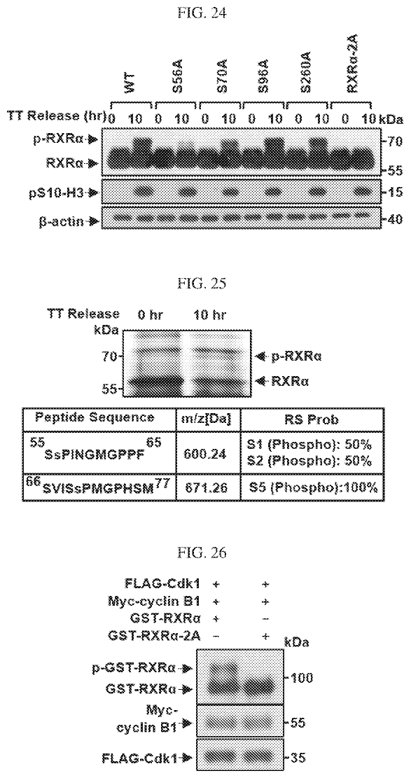

To identify Cdk1 phosphorylation sites in RXRα, it was first studied whether p-RXRα acts similar to other PLK1 partner proteins that bind to the PBD of PLK1 through their phosphate group. Cheng et al., EMBO J. 2003, 22, 5757-68; Elia et al., Science 2003, 299, 1228-31. Mutating either His538, Lys540, or both in the PBD of PLK1, two key residues responsible for PLK1 binding to the phosphate group of its interacting proteins, impaired PLK1 interaction with p-RXRα (FIG. 20). For RXRα, its A/B domain, which is responsible for p-RXRα interaction with PLK1 (FIGS. 1 to 5), was phosphorylated during mitosis (FIG. 21). Mutagenesis study revealed that amino acids 41 to 80 are critical for RXRα phosphorylation and interaction with PLK1 (FIGS. 22 and 23). There are four potential PLK1 docking motifs (S-pS/pT-P) in RXRα. Zitouni et al., Nat. Rev. Mol. Cell Biol. 2014, 15, 433-52. Replacing either Ser56 or Ser70 with Ala reduced RXRα shift in mitotic cells, while their simultaneous mutations produced a mutant (RXRα-2A) that failed completely to shift (FIG. 24). In contrast, mutating the other two Ser residues, Ser96 and Ser260, showed no effect. Phosphorylation of Ser56 and Ser70 was confirmed by tandem mass spectrometry (MS/MS) analysis (FIG. 25). In vitro, Cdk1/cyclin B1 was able to phosphorylate GST-RXRα but not GST-RXRα-2A (FIG. 26). Thus, Ser56 and Ser70 are responsible for RXRα phosphorylation by Cdk1.

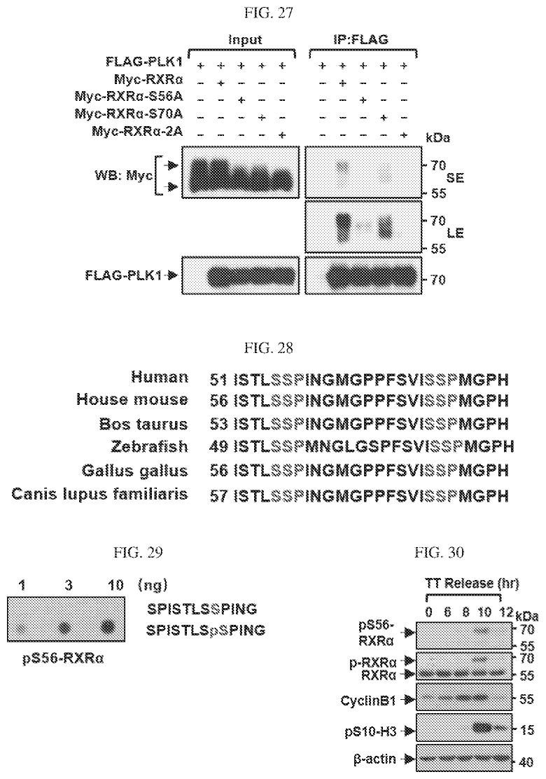

It was next determined whether Cdk1 phosphorylation of Ser56 and Ser70 serves as a PLK1 binding motif. Mutating Ser70 especially Ser56 strongly inhibited the ability of p-RXRαto interact with PLK1, while their simultaneous mutations (RXRα-2A) completely abolished the interaction (FIG. 27). For comparison, mutating Ser96 or Ser260 did not affect the interaction. A peptide encompassing pS56 docked well to the published phosphopeptide-binding cleft shaped by key amino acid residues from the PBD. Thus, Cdk1 phosphorylation of Ser56 and Ser70 mediates the interaction between p-RXRα and PLK1 during mitosis. Importantly, Ser56 and Ser70 and their surrounding amino acids are highly conserved across different species (FIG. 28), suggesting their evolutionarily conserved role in regulating RXRα activity.

### Example 5

### Effect of Cdk1 Phosphorylation of RXRα on its Translocation to the Centrosome

As the mitotic functions of PLK1 largely depend on its localization to various subcellular structures, it was examined the subcellular localization of RXRα during mitosis by immunostaining. Surprisingly, it was found that RXRα colocalized with γ-tubulin, a known centrosomal marker, during prophase of mitosis, although the receptor protein mainly resided in the nucleus at this stage of mitosis. RXRα association with the centrosome became very prominent during prometaphase and metaphase. After cells exit from mitosis, RXRα returned back to the nucleus. The immunostaining was specific as transfection of RXRα siRNA, which reduced RXRα expression, abrogated RXRα immunostaining at the centrosome. Furthermore. RXRα (mCherry-RXRα) or RXRα mutant lacking its DBD (mCherry-RXRα-ΔDBD) fused with mCherry localized to the centrosome during mitosis. Centrosome sedimentation analysis (Wigley et al., J. Cell. Biol. 1999, 145, 481-90) also demonstrated co-sedimentation of p-RXRα with γ-tubulin in centrosome fractions. Examination of the subcellular localization of RXRα and PLK1 revealed that RXRα colocalized with PLK1 only at the centrosome, but not other PLK1 localization sites such as kinetochores and central spindles. Transfected mCherry-RXRα also colocalized with PLK1 at the centrosome in mitotic cells. In situ proximity ligation assay (PLA) assay (Fredriksson et al., Nat. Biotechnol. 2002, 20, 473-77) confirmed their colocalization and direct interaction at the centrosome in prophase and metaphase cells but not in G2 cells. Collectively, these results demonstrated that RXRα, although recognized as a transcription factor, is a centrosomal PLK1-interacting protein during mitosis.

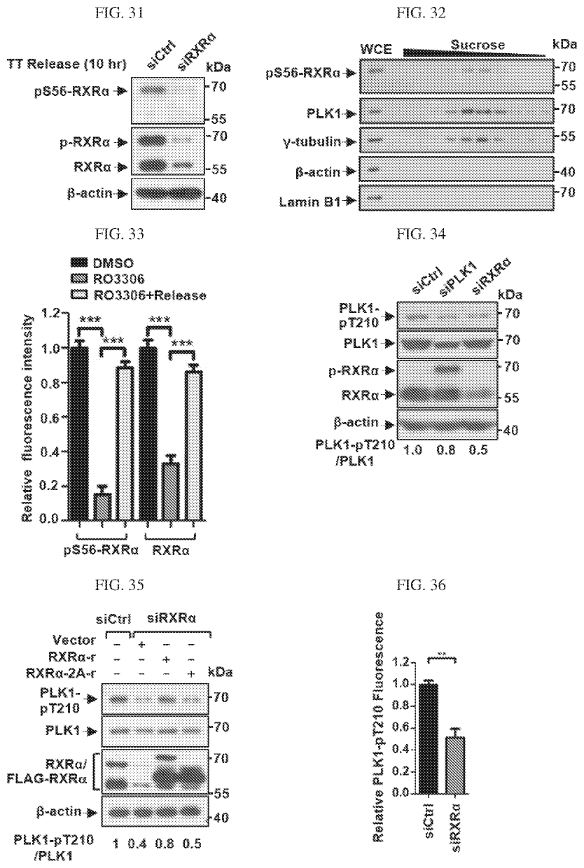

To characterize the role of Cdk1-dependent phosphorylation of RXRα in its translocation to the centrosome, it was generated an anti-RXRα antibody (anti-pS56-RXRα) that specifically recognizes Ser56 phosphorylated peptide (FIG. 29). Unlike regular anti-RXRα antibody that recognizes both RXRα and p-RXRα, the anti-pS56-RXRα antibody reacted only with p-RXRα but not RXRα (FIG. 30). The level of p-RXRα detected by the antibody was reduced by transfection of RXRα siRNA (FIG. 31) or treatment with RO-3306, revealing its specificity. By using the antibody, it was confirmed the phosphorylation of Ser56 of RXRα by Cdk1/cyclinB1 in vitro and the presence of p-RXRα at the centrosome and its centrosomal coaccumulation with PLK1 (FIG. 32). It also demonstrated that p-RXRα began to associate with the centrosome and colocalized with PLK1 at prophase, which became prominent at prometaphase and metaphase. The staining was specific as RO-3306 that inhibited RXRα shift eliminated centrosomal staining by the antibody, which could be rescued when cells were released again into mitosis (FIG. 33). RO-3306 also inhibited centrosomal RXRα staining by the regular anti-RXRα antibody. Thus, Cdk1-dependent phosphorylation is required for RXRα translocation to the centrosome. This was elaborated by data showing that substitution of Ser56 and Ser70 in RXRα with Ala (RXRα-2A), but not with Asp (RXRα-2D) impaired its translocation to the centrosome.

In studying how Cdk1 phosphorylation of RXRα promotes its centrosomal translocation, it was found that RXRα-LBD, a mutant incapable of binding PLK1, resided at the centrosome. Mutating PLK1-binding motif in RXRα-ΔDBD (RXRα-ΔDBD-2A) also failed to inhibit its centrosomal localization. Thus, the centrosomal localization of p-RXRα is mediated by its C-terminal LBD. This raised an intriguing possibility that Cdk1-dependent phosphorylation serves to activate the centrosomal targeting activity of RXRα-LBD, which is otherwise restrained before cells entering mitosis. It was previously reported that there is an intramolecular interaction between the N-terminal and C-terminal regions in RXRα. Chen et al., Nat. Commun. 2017, 8, 16066. Given that Cdk1 phosphorylation causes a major RXRα conformation change, it was asked whether Cdk1-mediated phosphorylation disrupts the intramolecular interaction, leading to the exposure of RXRα-LBD for targeting the centrosome. Indeed, RXRα-LBD strongly interacted with the regular form of RXRα-1-235, but not its mobility-shifted form, revealing an inhibitory effect of Cdk1-mediated phosphorylation on RXRα intramolecular interaction.

### Example 6

### Role of Cdk1 Phosphorylation of RXRα in PLK1 Activation

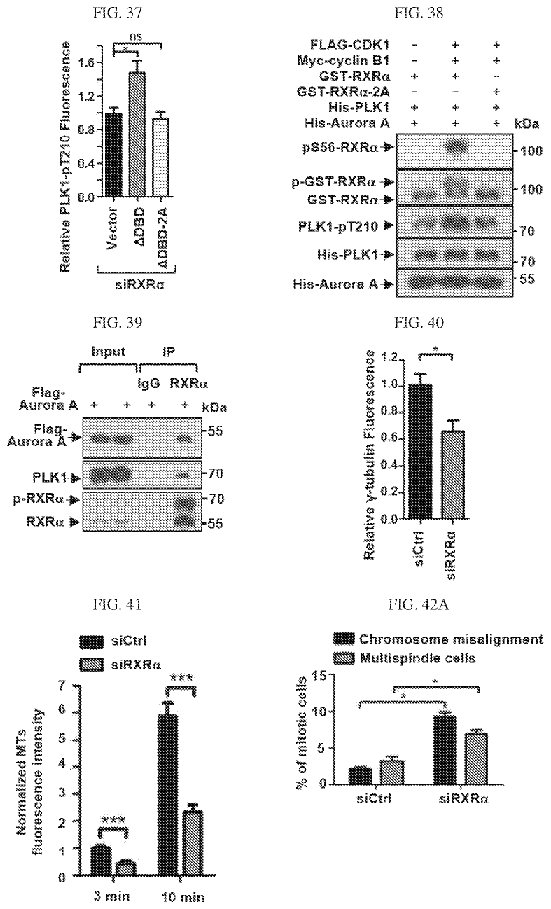

PLK1 is activated by phosphorylation at Thr210 during mitosis by Aurora A. Macurek et al., Nature 2008, 455, 119-23; Joukov and De Nicolo, Sci. Signal. 2018, 11, eaar4195. The level of PLK1 phosphorylation correlated well with that of p-RXRα during mitosis. Transfection of RXRα siRNA significantly reduced the phosphorylation of endogenous PLK1 (FIG. 34) and transfected Flag-PLK1 in mitotic cells, which was rescued by transfection of siRNA-resistant RXRα (RXRα-r) but not RXRα-2A (RXRα-2A-r) (FIG. 35). Similar to the effect of RXRα depletion, stable or transient transfection of RXRα-2A inhibited PLK1 phosphorylation, likely due to its dominant-negative effect. Inhibition of RXRα phosphorylation by RO-3306 also reduced PLK1 phosphorylation in a dose dependent manner. Furthermore, transfecting RXRα siRNA reduced PLK1-pT210 staining at the centrosome in prophase or metaphase cells (FIG. 36), which was compromised by transfection of RXRα-ΔDBD but not RXRα-ΔDBD-2A (FIG. 37), while it had no effect on the staining of PLK1 and centrin. The effect of RXRα on PLK1 activation was limited to the centrosomes, as knocking down RXRα did not inhibit PLK1-pT210 staining at the kinetochores (FIG. 36) and central spindles.

To study how Cdk1-induced p-RXRα interaction with PLK1 enhanced PLK1 activation, it was tested whether it enhanced PLK1 phosphorylation by Aurora A. Thus, GST-RXRα was first subjected to Cdk1/cyclin B1 phosphorylation in vitro. After eliminating Cdk1 and cyclin B1, it was incubated with His-PLK1 and His-Aurora A. The ability of Aurora A to phosphorylate PLK1 was significantly enhanced by Cdk1-phosphorylated GST-p-RXRα, but not GST-RXRα or GST-RXRα-2A (FIG. 38). Transfected RXRα abolished the intramolecular interaction of PLK1 and interacted not only with PLK1 but also Aurora A (FIG. 39). Thus, Cdk1-induced p-RXRα translocation to the centrosome likely serves to enhance Aurora A phosphorylation of PLK1 by mitigating the auto-suppression of PLK1.

### Example 7

### Role of p-RXRα Interaction with PLK1 in Centrosome Maturation and Function

At the onset of mitosis, the centrosome undergoes maturation characterized by a dramatic expansion of the pericentriolar material (PCM) and a robust increase in MT-organization activity. Immunostaining of mitotic HeLa cells revealed that the staining intensity of γ-tubulin (FIG. 40) and the scaffold protein Cep192 were reduced during prophase or metaphase when cells were transfected with RXRα siRNA. In contrast, accumulation of centrin, a centriole marker, was barely affected (FIG. 40). MT regrowth assay showed that the density of microtubules nucleating from centrosomes of RXRα siRNA-transfected prophase cells (FIG. 41) and metaphase cells was significantly decreased. Transfection of RXRα-ΔDBD but not RXRα-ΔDBD-2A into RXRα siRNA-transfected prophase cells could compromise the effect of RXRα depletion. Thus, p-RXRα plays a role in promoting centrosome maturation and MT nucleation.

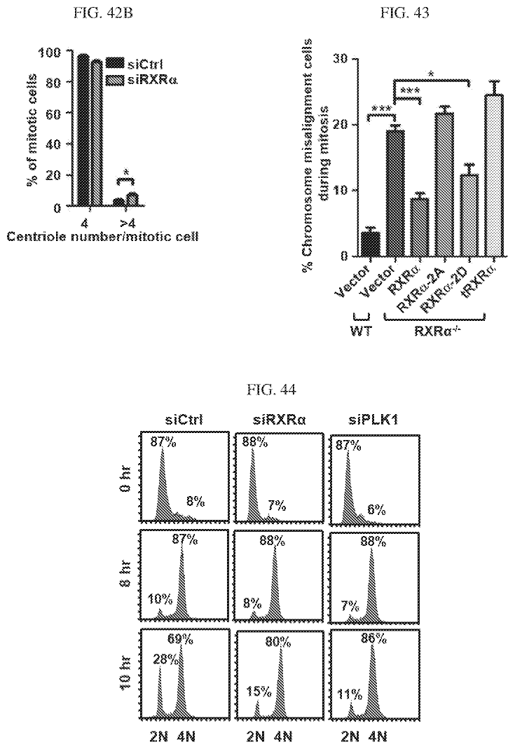

Proper centrosome maturation and nucleation are of crucial importance for the assembly of a bipolar mitotic spindle and subsequent faithful segregation of chromosomes into two daughter cells. RXRα-depleted mitotic cells displayed malformed bipolar spindles, severe chromosome misalignment and segregation defects as well as multicentrosomes (FIG. 42), reminiscent of those observed by PLK1 ablation. Induction of chromosome misalignment by RXRα depletion was rescued by retransfection of RXRα but not RXRα-2A. Depleting RXRα from cells by Crispr/Cas9 genome editing strategy also significantly increased the frequency of chromosome misalignment, which was again compromised by retransfection of RXRα and RXRα-2D, but not RXRα-2A (FIG. 43). Depleting RXRα also increased the number of cells with multispindles and multicentrosomes. Thus, p-RXRα is involved in modulating proper bipolar spindle assembly and chromosome segregation during mitosis.

### Example 8

### Role of p-RXRα Interaction with PLK1 in Mitotic Progression

The centrosome is important not only for microtubule organization but also for mitosis progression. Transfection of RXRα siRNA delayed the completion of mitosis, coincident with delayed cyclin B1 degradation upon transfection of RXRα siRNA or RXRα-2A. It also decreased the number of HeLa cells re-entering to G1 phase (from 28% to 15%), similar to the effect of PLK1 siRNA transfection (FIG. 44). The inhibitory effect of RXRα siRNA transfection was attenuated by re-expression of RXRα but not RXRα-2A. Overexpression of RXRα facilitated while transfection of RXRα-2A delayed the mitotic progression. The production of binucleate and multinucleate cells was enhanced by transfection of RXRα siRNA (from 4.4% to 18.6%) or depleting RXRα by CRISPR/Cas9 (from 5.99% to 23.1%), which was attenuated by transfection of RXRα but not RXRα-2A. These data are in agreement with the role of RXRα and PLK1 in the production of binucleate and multinucleate cells. Thus, p-RXRα interaction with PLK1 also modulates mitotic progression and cytokinesis.

### Example 9

### Levels of p-RXRα in Cancer Cells and Tumor Tissues

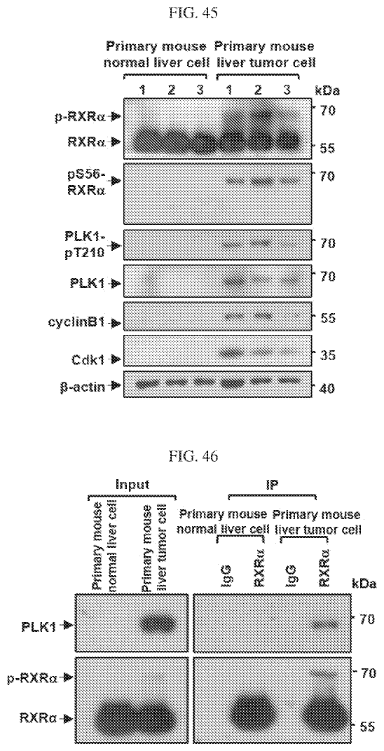

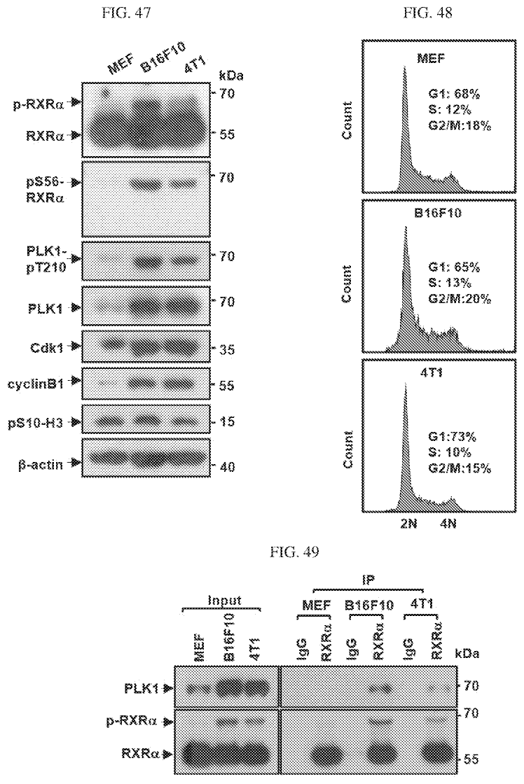

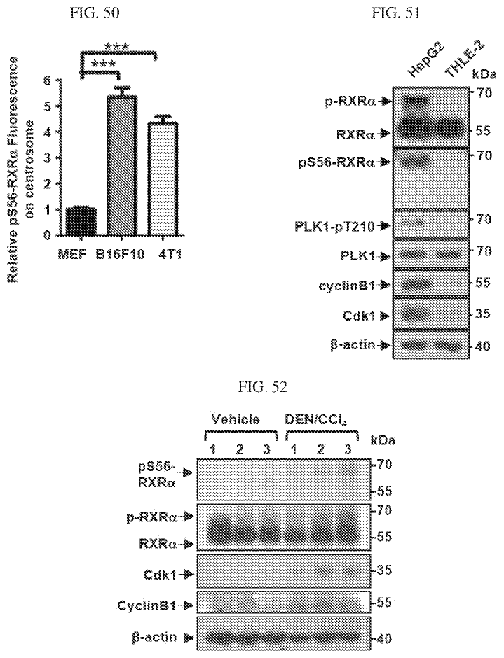

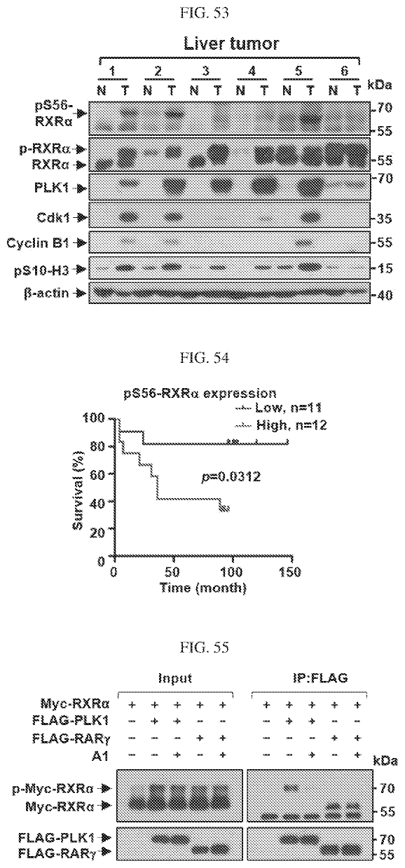

The finding that p-RXRα promotes PLK1 activation and mitotic progression led to study whether it is abnormally elevated in cancer cells. Thus, primary hepatocytes were from normal or tumor livers from mice fed with normal chow or high-fat high-cholesterol diet (HFHC) that induces spontaneous liver tumor development. p-RXRα was detected in primary liver tumor, but not normal liver cells, while RXRα was similarly expressed in both cell types (FIG. 45). In primary liver tumor cells, the expression of p-RXRα correlated positively with the activation of Cdk1 and PLK1. It interacted (FIG. 46) and colocalized with PLK1 at the centrosome. It was compared the expression of p-RXRα in B16F10 melanoma and 4T1 mammary carcinoma with noncancerous mouse embryonic fibroblast (MEF) cells, all of which underwent similar cell cycle progression (FIG. 48). Again, p-RXRα was highly expressed in B16F10 and 4T1 but not MEF cells (FIG. 47), where it interacted (FIG. 49) and colocalized (FIG. 50) with PLK1 at the centrosome. p-RXRα was also detected in various liver cancer but not THLE-2 and QSG-7701 noncancerous liver cell lines (FIG. 51). The tumor selective expression of p-RXRα was further illustrated by its expression in liver tumor but not normal liver tissues prepared from mice injected with diethylnitrosamine (DEN) followed by repeated administration of carbon tetrachloride (CCl₄), which again correlated positively with Cdk1 activation (FIG. 52). The expression of p-RXRα is likely clinically-relevant, as its levels are highly elevated in tumor tissues (T) compared to their corresponding tumor adjacent normal tissues (N) from patients with liver cancer and colorectal cancer (FIG. 53). As shown in Table 1, detailed analysis of additional 60 patients with hepatocellular carcinoma by a Chi-square test revealed a close correlation between pS56-RXRα expression and Cdk1 expression (p=0.00006) or PLK1 activation (p=0.00035). The level of pS56-RXRα also positively correlated with the presence of cancer embolus in the portal vein (p=0.00030) (Table 1), a risk factor associated with poor survival rate of liver cancer patients. Significantly, the survival time of patients was negatively correlated with the expression of pS56-RXRα (p=0.0321) (FIG. 54). Thus, p-RXRα and its interaction with PLK1 may play a role in mediating the effect of Cdk1 on activating PLK1 and promoting tumor cell proliferation.

TABLE 1
Correlation Between Clinicopathological Parameters
and pS56-RXRα Low or High Expression

| | | | pS56-RXRα expression | | |
|---|---|---|---|---|---|
| Parameters | Category | Cases | Low | High | P |
| Sex | Male | 43 | 20 | 23 | 0.03556 |
| | Female | 17 | 13 | 4 | |
| Age | ≥60 | 18 | 10 | 8 | 0.95632 |
| | <60 | 42 | 23 | 19 | |
| Tumor size | ≥5 cm | 28 | 17 | 11 | 0.40515 |
| | <5 cm | 32 | 16 | 16 | |
| Cancer embolus | Present | 36 | 15 | 21 | 0.00030 |
| | Absent | 17 | 16 | 1 | |
| Five year survival | Live | 9 | 6 | 3 | 0.03967 |
| | Dead | 10 | 2 | 8 | |
| Cdk1 expression | Low | 18 | 17 | 1 | 0.00006 |
| | High | 42 | 16 | 26 | |
| PLK-pThr210 | Low | 33 | 25 | 8 | 0.00035 |
| expression | High | 27 | 8 | 19 | |

### Example 10

### Selective Inhibition of the Interaction of p-RXRα with PLK1 by an RXRα Ligand

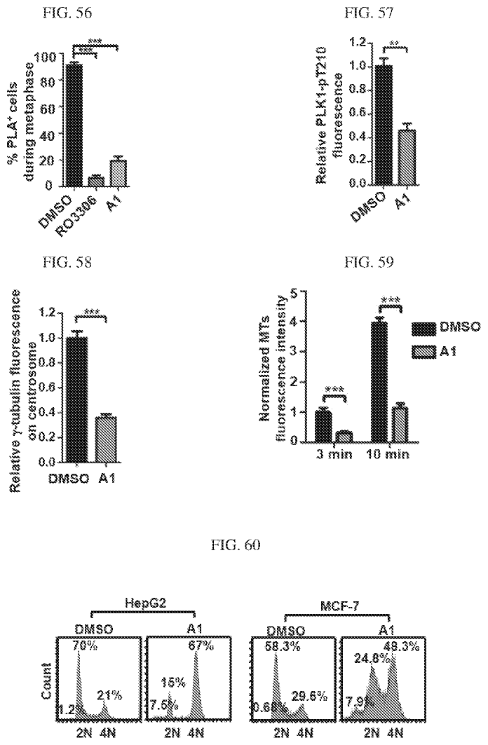

The finding that p-RXRα interaction with PLK1 occurs in cancer but not normal cells provides an opportunity of developing tumor-selective RXRα therapeutics targeting the interaction. Although classical ligands such as 9-cis-RA showed no apparent effect on the interaction (FIG. 1), several non-canonical ligands were identified to be effective on inducing mitosis arrest, similar to the effect of the PLK1 inhibitor BI2536. Compound 1A inhibited p-RXRα interaction with PLK1 but had no effect on RXRα heterodimerization with RARγ (FIG. 55). Compound A1 inhibited the in situ interaction between p-RXRα and PLK1 at the centrosome (FIG. 56), similar to the effect of RO-3306. As the results, compound A1 reduced the level of PLK1-pT210 (FIG. 57) but not PLK1 at the centrosome, suppressed centrosome maturation (FIG. 58), inhibited α-tubulin nucleation (FIG. 59), and caused centrosomal aberrations, reminiscent of the effects of RXRα depletion (FIGS. 40 to 42) and PLK1 inhibition (Gumireddy et al., Cancer Cell 2005, 7, 275-86; Steegmaier et al., Curr. Biol. 2007, 17, 316-22; Reindl et al., Chem. Biol. 2008, 15, 459-66). Thus, compound A1 suppresses centrosomal activities by inhibiting the p-RXRα/PLK1 interaction.

### Example 11

### Induction of Mitotic Arrest and Mitotic Catastrophe of Cancer Cells by Compound A1

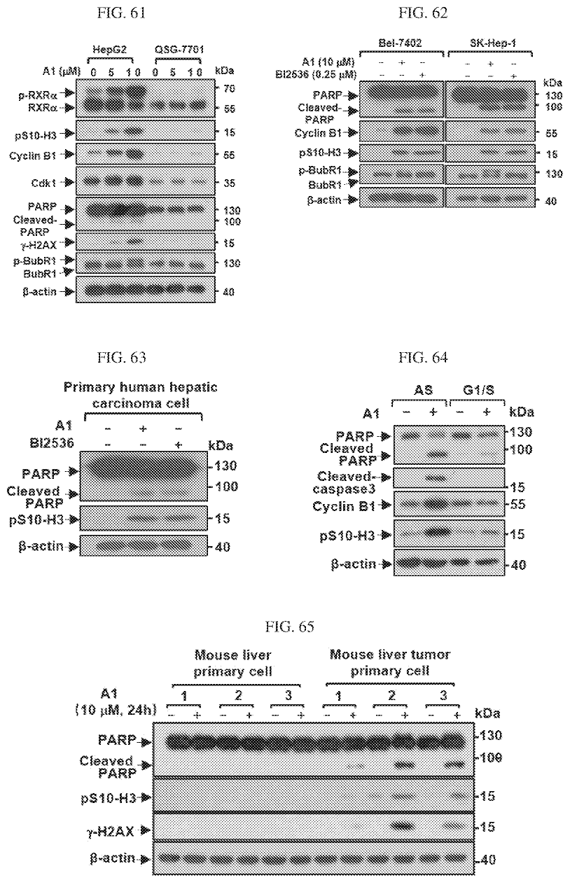

Consistent with its inhibition of centrosomal activities, compound A1, like PLK1 inhibitors B12536 and poloxin, inhibited mitotic progression of cancer cells. The inhibitory effect of compound A1 was RXRα dependent and observed in many other cancer cell lines (FIG. 60). The severe mitotic failure can be a causal factor that initiates the cell death program. Vitale et al., Nat. Rev. Mol. Cell Biol. 2011, 12, 385-92. Indeed, compound A1, induced apoptosis of cancer cells in a dose-dependent and RXRα-dependent manner. Induction of mitotic arrest and apoptosis by compound A1 was observed in various cancer cell lines (FIGS. 61 and 62), primary human hepatocellular carcinoma cells (FIG. 63), and primary mouse liver tumor cells (FIG. 65). Prolonged mitotic arrest can lead to cell death via mitotic catastrophe. Id. Upon treatment with compound A1, synchronized HeLa cells progressed through S phase normally and arrested in mitotic phase, which was followed by apoptosis. Compound A1 is also more effective in asynchronous cells (AS) than in synchronized GUS cells (FIG. 64). Thus, compound A1-induced apoptosis occurred in mitotic but not interphase cells. Compound A1-induced apoptosis was accompanied with the activation of the spindle assembly checkpoint (SAC), indicated by the expression of the mobility-shifted band of mitotic check point protein BubR1 (FIGS. 61, 62, and 68. Cancer cells treated with compound A1 exhibited multi-lobular, giant nuclear and/or highly fragmented/bursted nuclei, characteristics of mitotic catastrophe. Collectively, these results demonstrated that compound A1 induces chromosome misalignment, aberrant mitotic spindles, SAC activation, and mitotic arrest, leading to mitotic catastrophe of cancer cells.

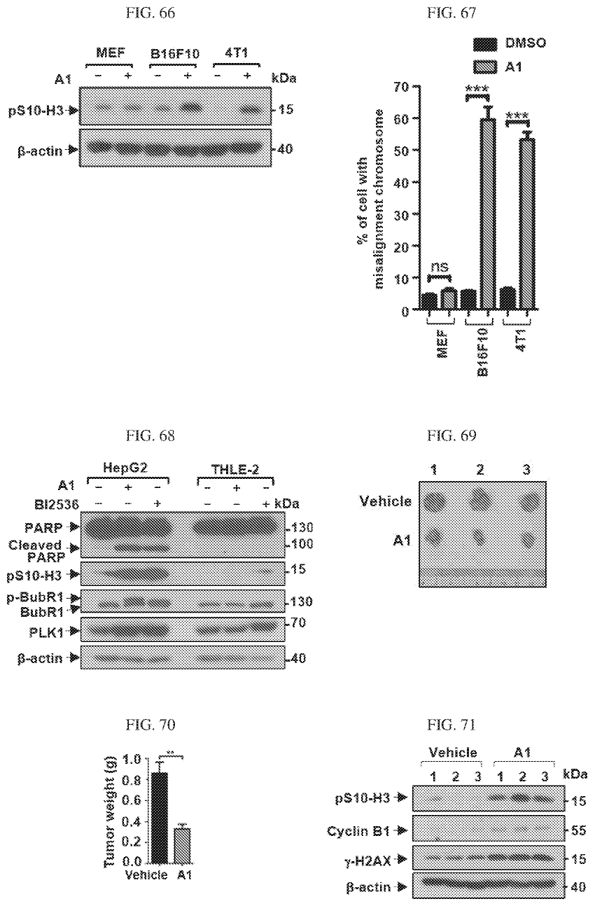

It was next studied the hypothesis that tumor cells could become adapted to p-RXRα expression for their proliferation, thus making them more vulnerable than normal cells to inhibition by compound A1. compound A1 treatment induced G2/M arrest, DNA damage response, and apoptosis in primary liver cancer but not normal cells (FIG. 65). It caused mitotic arrest (FIG. 66) and chromosomal aberrations (FIG. 67) in tumor but not noncancerous MEF cells. The lack of compound A1 effects in primary normal liver cells and MEFs was not due to reduced level of PLK1 expression (FIGS. 45 and 47), as it also exhibited differential effects on inducing G2/M arrest, SAC activation, and apoptosis in HepG2 liver cancer and THLE-2 nontumorous liver cells, both of which expressed similar levels of PLK1 (FIG. 68). Compound A1 showed potent effect in p-RXRα-positive HepG2 liver cancer but not p-RXRα-negative noncancerous QSG-7701 cells, whereas BI2536 was active even in QSG-7701 cells. Thus, unlike BI2536 that targets PLK1, compound A1 targets p-RXRα-PLK1 interaction, which is a tumor selective event. To test compound A1 in vivo, it was administered to nude mice bearing subcutaneously implanted HepG2 xenografts. Compound A1 strongly inhibited the growth of HepG2 tumor without any signs of overt toxicities (FIGS. 69 and 70). The inhibitory effect of compound A1 was due to its induction of mitotic arrest, chromosome aberrations, DNA damage response, and caspase 3 activation in tumor cells (FIG. 71). Significantly, the effects of compound A1 were not observed in normal liver tissues. In addition, compound A1 had no effect on mitotic arrest and DNA damage response in normal tissues of kidney, intestine, spleen, and heart. Thus, compound A1 selectively induces mitotic arrest, mitotic aberrations, DNA damage responses, SAC activation, and ultimately causes mitotic catastrophe of cancer but not normal cells.

Sequences described herein are provided in the sequence table below.

SEQUENCE TABLE

| SEQ ID NO: | Description | Amino Acid Sequence |
|---|---|---|
| 1 | Human RXRα | MDTKHFLPLDFSTQVNSSLTSPTGRGSMAAPSLH |
| | (pSer 56) | PSLGPGIGSPGQLHSPISTLS(pS)PINGMGPPFSVISS |
| | | PMGPHSMSVPTTPTLGFSTGSPQLSSPMNPVSSSE |
| | | DIKPPLGLNGVLKVPAHPSGNMASFTKHICAICG |
| | | DRSSGKHYGVYSCEGCKGFFKRTVRKDLTYTCR |
| | | DNKDCLIDKRQRNRCQYCRYQKCLAMGMKREA |
| | | VQEERQRGKDRNENEVESTSSANEDMPVERILEA |
| | | ELAVEPKTETYVEANMGLNPSSPNDPVTNICQAA |
| | | DKQLFTLVEWAKRIPHFSELPLDDQVILLRAGWN |
| | | ELLIASFSHRSIAVKDGILLATGLHVHRNSAHSAG |
| | | VGAIFDRVLTELVSKMRDMQMDKTELGCLRAIV |
| | | LFNPDSKGLSNPAEVEALREKVYASLEAYCKHK |
| | | YPEQPGRFAKLLLRLPALRSIGLKCLEHLFFFKLIG |
| | | DTPIDTFLMEMLEAPHQMT |
| 2 | Human RXRα | MDTKHFLPLDFSTQVNSSLTSPTGRGSMAAPSLH |
| | (Unphosphorylated | PSLGPGIGSPGQLHSPISTLSSPINGMGPPFSVISSP |
| | Ser 56) | MGPHSMSVPTTPTLGFSTGSPQLSSPMNPVSSSED |
| | | IKPPLGLNGVLKVPAHPSGNMASFTKHICAICGDR |
| | | SSGKHYGVYSCEGCKGFFKRTVRKDLTYTCRDN |
| | | KDCLIDKRQRNRCQYCRYQKCLAMGMKREAVQ |
| | | EERQRGKDRNENEVESTSSANEDMPVERILEAEL |
| | | AVEPKTETYVEANMGLNPSSPNDPVTNICQAADK |
| | | QLFTLVEWAKRIPHFSELPLDDQVILLRAGWNEL |
| | | LIASFSHRSIAVKDGILLATGLHVHRNSAHSAGVG |
| | | AIFDRVLTELVSKMRDMQMDKTELGCLRAIVLF |
| | | NPDSKGLSNPAEVEALREKVYASLEAYCKHKYP |
| | | EQPGRFAKLLLRLPALRSIGLKCLEHLFFFKLIGDT |
| | | PIDTFLMEMLEAPHQMT |
| 3 | Phosphopeptide | SPISTLS(pS)PING |
| 4 | Unphosphorylated | SPISTLSSPING |
| | Peptide | |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A retinoid X receptor alpha (RXRα) binder that specifically binds to an epitope of an RXRα, wherein the epitope comprises a phosphorylated serine at position 56 or 70.

2. The RXRα binder of claim 1, wherein the RXRα is a human RXRα.

3. The RXRα binder of claim 1 or 2, wherein the RXRα has an amino acid sequence of SEQ ID NO: 1.

4. The RXRα binder of any one of claims 1 to 3, wherein the epitope comprises a phosphorylated serine at position 56.

5. The RXRα binder of any one of claims 1 to 4, wherein the epitope is a linear epitope.

6. The RXRα binder of any one of claims 1 to 5, wherein the epitope has a length ranging from about 5 to about 50 amino acids.

7. The RXRα binder of any one of claims 1 to 6, wherein the epitope has a length of about 8, about 9, about 10, about 11, about 12, about 13, about 14, about 15, about 16, about 17, about 18, about 19, or about 20 amino acids.

8. The RXRα binder of any one of claims 1 to 7, wherein the epitope comprises an amino acid sequence that is no less than about 80% identical to the amino acid sequence of SEQ ID NO: 3.

9. The RXRα binder of any one of claims 1 to 8, wherein the epitope comprises an amino acid sequence of SEQ ID NO: 3.

10. The RXRα binder of any one of claims 1 to 9, wherein the RXRα binder has a selectivity for an RXRα comprising an amino acid sequence of SEQ ID NO: 1 over an RXRα comprising an amino acid sequence of SEQ ID NO: 2.

11. The RXRα binder of claim 10, wherein the selectivity is no greater than about 0.1.

12. The RXRα binder of any one of claims 1 to 11, wherein the RXRα binder is an antibody or an antigen-binding fragment thereof.

13. The RXRα binder of claim 12, wherein the antibody is a monoclonal antibody or an antigen-binding fragment thereof.

14. The RXRα binder of claim 12, wherein the antibody is a polyclonal antibody or an antigen-binding fragment thereof.

15. The RXRα binder of any one of claims 12 to 14, wherein the antibody is an IgG.

16. An immunogenic composition comprising a phosphopeptide that comprises an amino acid sequence of an epitope of an RXRα, and optionally an adjuvant; wherein the epitope comprises a phosphorylated serine at position 56 or 70.

17. The immunogenic composition of claim 16, wherein the RXRα is a human RXRα.

18. The immunogenic composition of claim 16 or 17, wherein the RXRα has an amino acid sequence of SEQ ID NO: 1.

19. The immunogenic composition of any one of claims 16 to 18, wherein the epitope comprises a phosphorylated serine at position 56.

20. The immunogenic composition of any one of claims 16 to 19, wherein the epitope is a linear epitope.

21. The immunogenic composition of any one of claims 16 to 20, wherein the epitope has a length ranging from about 5 to about 50 amino acids.

22. The immunogenic composition of any one of claims 16 to 21, wherein the epitope has a length of about 8, about 9, about 10, about 11, about 12, about 13, about 14, about 15, about 16, about 17, about 18, about 19, or about 20 amino acids.

23. The immunogenic composition of any one of claims 16 to 22, wherein the epitope comprises an amino acid sequence that is no less than about 80% identical to the amino acid sequence of SEQ ID NO: 3.

24. The immunogenic composition of any one of claims 16 to 23, wherein the phosphopeptide has an amino acid sequence of SEQ ID NO: 3.

25. A method of detecting a phosphorylated RXRα in a biological sample, comprising the steps of:
contacting the biological sample with the RXRα binder of any one of claims 1 to 15 to form an RXRα binder/phosphorylated RXRα complex; and
detecting the RXRα binder/phosphorylated RXRα complex.

26. The method of claim 25, further comprising a step of obtaining the biological sample from a subject.

27. A method of diagnosing a proliferative disease in a subject by detecting the level of a phosphorylated RXRα in a biological sample from the subject, comprising the steps of:
contacting the biological sample with the RXRα binder of any one of claims 1 to 15 to form an RXRα binder/phosphorylated RXRα complex; and
detecting the RXRα binder/phosphorylated RXRα complex.

28. A method of screening a subject for a proliferative disease by detecting the level of a phosphorylated RXRα in a biological sample from the subject, comprising the steps of:
contacting the biological sample with the RXRα binder of any one of claims 1 to 15 to form an RXRα binder/phosphorylated RXRα complex; and
detecting the RXRα binder/phosphorylated RXRα complex.

29. The method of claim 27 or 28, further comprising a step of obtaining the biological sample from the subject.

30. The method of any one of claims 25 to 29, wherein the subject is a human.

31. The method of any one of claims 25 to 30, wherein the biological sample is a blood, plasma, serum, cerebral spinal fluid, mucus, saliva, semen, sputum, stool, or urine sample.

32. The method of any one of claims 25 to 30, wherein the biological sample is a biopsy of a tissue.

33. The method of any one of claims 25 to 32, wherein the detecting step is performed visually, colorimetrically, fluorescently, by chemiluminescence, by electrochemiluminescence, radioactively, or using a biosensor.

34. The method of any one of claims 25 to 33, wherein the RXRα binder is immobilized onto a surface of a solid phase.

35. The method of claim 34, wherein the solid phase is a biosensor.

36. The method of claim 34 or 35, wherein the solid phase is an SPR or BLI biosensor.

37. The method of any one of claims 34 to 36, wherein the method is performed in the format of an SPR or BLI immunoassay.

38. The method of claim 34, comprising the steps of:
contacting the biological sample with the RXRα binder of any one of claims 1 to 15 to form an RXRα binder/phosphorylated RXRα complex, wherein the RXRα binder is immobilized onto the surface of the solid phase;
contacting the RXRα binder/phosphorylated RXRα complex with a detection agent to form a detectable complex; and
detecting the detectable complex.

39. The method of claim 38, wherein the detection agent is a detection antibody.

40. The method of claim 39, wherein the detection antibody is specific to an RXRα.

41. The method of claim 40, wherein the detection antibody does not compete with the RXRα binder of any one of claims 1 to 15 for binding to a phosphorylated RXRα of SEQ ID NO: 1.

42. The method of any one of claims 39 to 41, wherein the detection antibody is a monoclonal antibody or an antigen-binding fragment thereof.

43. The method of any one of claims 39 to 41, wherein the detection antibody is a polyclonal antibody or an antigen-binding fragment thereof.

44. The method of any one of claims 39 to 43, wherein the detection antibody is a chicken, donkey, goat, guinea pig, hamster, mouse, rabbit, rat, or sheep antibody.

45. The method of any one of claims 39 to 44, wherein the detection antibody comprises a reporter.

46. The method of any one of claims 39 to 45, wherein the detection antibody is an enzyme conjugated secondary antibody.

47. The method of claim 46, wherein the detection antibody is conjugated with a peroxidase.

48. The method of claim 46 or 47, wherein the detection antibody is conjugated with a horseradish peroxidase or alkaline peroxidase.

49. The method of any one of claims 38 to 48, wherein the solid phase is a membrane or a well in a microplate.

50. The method of any one of claims 38 to 49, wherein the method is performed in the format of an enzyme linked immunosorbent assay.

51. The method of any one of claims 25 to 33, comprising the steps of:
contacting a biological sample from a subject with the RXRα binder of any one of claims 1 to 15 to form an RXRα binder/phosphorylated RXRα complex, wherein the RXRα binder comprises a reporter;
contacting the RXRα binder/phosphorylated RXRα complex with a capture agent to capture the RXRα binder/phosphorylated RXRα complex to form a detectable complex, wherein the capture agent is immobilized onto a surface of a membrane; and
detecting the detectable complex.

52. The method of claim 51, wherein the capture agent is a capture antibody.

53. The method of claim 52, wherein the capture antibody is specific to an RXRα.

54. The method of claim 53, wherein the capture antibody does not compete with the RXRα binder of any one of claims 1 to 15 when binding to a phosphorylated RXRα of SEQ ID NO: 1.

55. The method of any one of claims 52 to 54, wherein the capture antibody is a monoclonal antibody or an antigen-binding fragment thereof.

56. The method of any one of claims 52 to 54, wherein the capture antibody is a polyclonal antibody or an antigen-binding fragment thereof.

57. The method of any one of claims 52 to 56, wherein the capture antibody is a chicken, donkey, goat, guinea pig, hamster, mouse, rabbit, rat, or sheep antibody.

58. The method of any one of claims 51 to 57, wherein the reporter is a colorimetric reporter.

59. The method of any one of claims 51 to 58, wherein the reporter is a colorimetric particle.

60. The method of any one of claims 51 to 59, wherein the reporter is a gold or latex particle.

61. The method of any one of claims 51 to 60, wherein the method is performed in the format of a lateral flow assay.

62. A method of treating, preventing, or ameliorating one or more symptoms of a proliferative disease in a subject, comprising administering a therapeutically effective amount of a retinoid X receptor alpha/polo-like kinase 1 (RXRα/PLKl) modulator that inhibits the interaction of a PLK1 with an RXRα comprising a phosphorylated serine at position 56 or 70.

63. The method of claim 62, wherein the proliferative disease is cancer.

64. The method of claim 63, wherein the cancer is a solid tumor.

65. The method of claim 63 or 64, wherein the cancer is breast cancer, cervical cancer, colorectal cancer, cutaneous squamous cell carcinoma (CSCC), endometrial carcinoma, esophageal cancer, gastric cancer, head and neck squamous cell cancer (HNSCC), hepatocellular carcinoma (HCC), Hodgkin lymphoma, melanoma, Merkel cell carcinoma (MCC), a microsatellite instability cancer, a mismatch repair deficient cancer, non-small cell lung cancer (NSCLC), primary mediastinal large B-cell lymphoma (PMBCL), renal cell carcinoma (RCC), small cell lung cancer (SCLC), or urothelial cancer (UC).

66. The method of claim 63, wherein the cancer is leukemia.

67. The method of claim 66, wherein the cancer is acute lymphocytic leukemia (ALL), acute myeloid leukemia (AML), chronic lymphocytic leukemia (CLL), or chronic myeloid leukemia (CML).

68. The method of any one of claims 63 to 67, wherein the cancer is relapsed and/or refractory.

69. The method of any one of claims 63 to 68, wherein the cancer is drug resistant.

70. The method of any one of claims 63 to 69, wherein the cancer is metastatic.

71. The method of any one of claims 62 to 70, wherein the subject is a human.

72. A method of inhibiting the growth of a cell, comprising contacting the cell with an effective amount of an RXRα/PLK1 modulator that inhibits the interaction of a PLK1 with an RXRα comprising a phosphorylated serine at position 56 or 70.

73. A method of inducing apoptosis in a cell, comprising contacting the cell with an effective amount of an RXRα/PLK1 modulator that inhibits the interaction of a PLK1 with an RXRα comprising a phosphorylated serine at position 56 or 70.

74. A method of inhibiting mitotic progression in a cell, comprising contacting the cell with an effective amount of an RXRα/PLK1 modulator that inhibits the interaction of a PLK1 with an RXRα comprising a phosphorylated serine at position 56 or 70.

75. The method of any one of claims 72 to 74, wherein the cell is a cancerous cell.

76. The method of any one of claims 72 to 75, wherein the cell is a human cancerous cell.

77. The method of any one of claims 62 to 76, wherein the RXRα/PLK1 modulator inhibits the interaction of a PLK1 with an RXRα comprising a phosphorylated serine at position 56 as set forth in SEQ ID NO: 1.

78. The method of any one of claims 62 to 77, wherein the RXRα/PLK1 modulator is E)-N′-((2-hydroxynaphthalen-1-yl)methylene)-2-(4-methoxyphenyl)acetohydrazide, or a tautomer, a mixture of two or more tautomers, or an isotopic variant thereof; or a pharmaceutically acceptable salt, solvate, hydrate, or prodrug thereof.
